(19) United States
(12) Patent Application Publication
(10) Pub. No.: US 2024/0050099 A1
(43) Pub. Date: Feb. 15, 2024

(54) OCCLUSIVE DEVICES FOR TREATING VASCULAR DEFECTS AND ASSOCIATED SYSTEMS AND METHODS

(71) Applicant: Covidien LP, Mansfield, MA (US)

(72) Inventors: Robert A. Pecor, Aliso Viejo, CA (US); Junwei Li, Irvine, CA (US); Jared Akira Shimizu, Tustin, CA (US); John M. Wainwright, Foothill Ranch, CA (US); Elijah Klar, Dana Point, CA (US); Taylor S. Dorans, Rancho Santa Margarita, CA (US); Mehdi Matteo Rashidi, Irvine, CA (US)

(21) Appl. No.: 18/361,760

(22) Filed: Jul. 28, 2023

Related U.S. Application Data
(60) Provisional application No. 63/369,936, filed on Jul. 30, 2022.

(51) Int. Cl. A61B 17/12

(52) U.S. Cl. CPC A61B 17/12172; A61B 17/12113; A61B 17/12177

(57) ABSTRACT

Systems, methods, and devices for treating vascular defects are disclosed herein. In some embodiments, a device for treating an aneurysm includes a plurality of braided filaments configured to be implanted in an aneurysm cavity.

### CROSS-REFERENCE TO RELATED APPLICATION(S)

The present application claims the benefit of priority to U.S. Provisional Patent Application No. 63/369,936, filed Jul. 30, 2022, which is incorporated by reference herein in its entirety.

### TECHNICAL FIELD

The present technology generally relates to medical devices, and in particular, to occlusive devices for treating vascular defects and associated systems and methods.

### BACKGROUND

An intracranial aneurysm is a portion of an intracranial blood vessel that bulges outward from the blood vessel's main channel. This condition often occurs at a portion of a blood vessel that is abnormally weak because of a congenital anomaly, trauma, high blood pressure, or for another reason. Once an intracranial aneurysm forms, there is a significant risk that the aneurysm will eventually rupture and cause a medical emergency with a high risk of mortality due to hemorrhaging. When an unruptured intracranial aneurysm is detected or when a patient survives an initial rupture of an intracranial aneurysm, vascular surgery is often indicated. One conventional type of vascular surgery for treating an intracranial aneurysm includes using a microcatheter to dispose a platinum coil within an interior volume of the aneurysm. Over time, the presence of the coil should induce formation of a thrombus. Ideally, the aneurysm's neck closes at the site of the thrombus and is replaced with new endothelial tissue. Blood then bypasses the aneurysm, thereby reducing the risk of aneurysm rupture (or re-rupture) and associated hemorrhaging. Unfortunately, long-term recanalization (i.e., restoration of blood flow to the interior volume of the aneurysm) after this type of vascular surgery occurs in a number of cases, especially for intracranial aneurysms with relatively wide necks and/or relatively large interior volumes.

Another conventional type of vascular surgery for treating an intracranial aneurysm includes deploying a flow diverter within the associated intracranial blood vessel. The flow diverter is often a mesh tube that causes blood to preferentially flow along a main channel of the blood vessel while blood within the aneurysm stagnates. The stagnant blood within the aneurysm should eventually form a thrombus that leads to closure of the aneurysm's neck and to growth of new endothelial tissue, as with the platinum coil treatment. One significant drawback of flow diverters is that it may take weeks or months to form aneurysmal thrombus and significantly longer for the aneurysm neck to be covered with endothelial cells for full effect. This delay may be unacceptable when risk of aneurysm rupture (or re-rupture) is high. Moreover, flow diverters typically require antiplatelet therapy to prevent a thrombus from forming within the main channel of the blood vessel at the site of the flow diverter. Antiplatelet therapy may be contraindicated shortly after an initial aneurysm rupture has occurred because risk of re-rupture at this time is high and antiplatelet therapy tends to exacerbate intracranial hemorrhaging if re-rupture occurs. For these and other reasons, there is a need for innovation in the treatment of intracranial aneurysms. Given the severity of this condition, innovation in this field has immediate life-saving potential.

### SUMMARY

The present technology is illustrated, for example, according to various aspects described below. These various aspects are provided as examples and do not limit the subject technology.
1. An occlusive device for treating an aneurysm, the device comprising:
a mesh configured to be implanted in an aneurysm and comprising a proximal end portion, a distal end portion, a longitudinal axis extending therebetween, the mesh defining an opening at the distal end portion and outer and inner layers that meet distally at the distal end portion of the mesh and proximally at the proximal end portion of the mesh via a securing means,
wherein the mesh is configured to transition between a collapsed state and an expanded unconstrained state,
wherein in the collapsed state, the outer and inner layers are compressed within the elongated shaft for delivery to the aneurysm,
wherein in the expanded unconstrained state:
   an inner surface of the outer layer and an inner surface of the inner layer are separated by a radial distance such that the inner surfaces of the outer and inner layers enclose a cavity, and
   wherein the inner surface of the inner layer comprises a first region extending from the opening proximally, wherein the first region concave towards the inner surface of the outer layer, wherein the inner surface of the inner layer comprises a second region extending from the first region, and
   wherein the second region is convex towards the inner surface of the outer layer.
2. An occlusive device for treating an aneurysm, the device comprising:
a mesh configured to be implanted in an aneurysm and having a proximal end portion, a distal end portion, and a longitudinal axis extending therebetween, the mesh further comprising an opening at the distal end portion and outer and inner layers that meet distally at the distal end portion of the mesh and proximally at the proximal end portion of the mesh via a securing means,
wherein the mesh comprises a collapsed state for delivery through an elongated shaft to the aneurysm and an expanded, unconstrained state, and wherein, in the expanded, unconstrained state:
   an inner surface of the outer layer and an inner surface of the inner layer are separated by a radial distance such that the inner surfaces of the outer and inner layers enclose a first cavity, and
   the outer layer defines a diameter that increases then decreases along the longitudinal axis of the mesh between the proximal and distal end portions.
3. An occlusive device for treating an aneurysm, the device comprising:
a mesh configured to be implanted in an aneurysm and having a proximal end portion, a distal end portion, and a longitudinal axis extending therebetween, the mesh further comprising an opening at the distal end portion and outer and inner layers that meet distally at the distal end portion of the mesh and proximally at the proximal end portion of the mesh via a securing means,
wherein the mesh comprises a collapsed state for delivery through an elongated shaft to the aneurysm and an expanded, unconstrained state, and wherein, in the expanded, unconstrained state:
   an inner surface of the outer layer and an inner surface of the inner layer are separated by a radial distance such that the inner surfaces of the outer and inner layers enclose a first cavity, and
   the outer layer defines a first diameter at the proximal end portion of the mesh, a second diameter at an intermediate portion of the mesh, and a third diameter at the distal end portion of the mesh, wherein the individual first and third diameters are less than the second diameter.
4. A method for treating an aneurysm, the method comprising:
positioning a distal end of an elongated shaft in an aneurysm cavity;
releasing an occlusive member from the elongated shaft into the aneurysm cavity, the occlusive member comprising outer and inner layers, wherein releasing the occlusive member allows the occlusive member to self-expand into a first expanded state in which inner surfaces of the inner and outer layers enclose an interior region having a first shape and an outer surface of the inner layer defines a cavity; and
delivering an embolic element into the cavity applying force to the outer surface of the inner layer causing the inner layer to move radially outwardly towards the outer layer and transforming the occlusive member into a second expanded state in which the interior region has a second shape different than the first shape and a volume that is less than a volume of the interior region in the first expanded state.
5. An occlusive device for treating an aneurysm, the device comprising:
a mesh configured to be implanted in an aneurysm and having a proximal end portion, a distal end portion, and a longitudinal axis extending therebetween, the mesh further comprising an opening at the distal end portion and outer and inner layers that meet distally at the distal end portion of the mesh and proximally at the proximal end portion of the mesh via a securing means,
wherein the mesh comprises a collapsed state for delivery through an elongated shaft to the aneurysm and an expanded, unconstrained state, and wherein, in the expanded, unconstrained state:
   an inner surface of the outer layer and an inner surface of the inner layer are separated by a radial distance such that the inner surfaces of the outer and inner layers enclose a cavity, and
   the inner surface of the inner layer extends proximally from the opening along a first region that is concave towards the inner surface of the outer layer and then along a second region that is convex towards the inner surface of the outer layer.
6. An occlusive device for treating an aneurysm, the device comprising:
a mesh configured to be implanted in an aneurysm and having a proximal end portion, a distal end portion, and a longitudinal axis extending therebetween, the mesh further comprising an opening at the distal end portion and outer and inner layers that meet distally at the distal end portion of the mesh and proximally at the proximal end portion of the mesh via a securing means,
wherein the mesh comprises a collapsed state for delivery through an elongated shaft to the aneurysm and an expanded, unconstrained state, and wherein, in the expanded, unconstrained state:
   an inner surface of the outer layer and an inner surface of the inner layer are separated by a radial distance such that the inner surfaces of the outer and inner layers enclose a first cavity, and
   the outer layer has a diameter that increases then decreases along the longitudinal axis of the mesh between the proximal and distal end portions.
7. A method for treating an aneurysm, the method comprising:
positioning a distal end of an elongated shaft in an aneurysm cavity;
releasing an occlusive member from the elongated shaft into the aneurysm cavity, the occlusive member comprising outer and inner layers, wherein releasing the occlusive member allows the occlusive member to self-expand into a first expanded state in which inner surfaces of the inner and outer layers enclose an interior region having a first shape and an outer surface of the inner layer defines a cavity; and
delivering an embolic element into the cavity, thereby forcing the inner layer radially outward towards the outer layer and transforming the occlusive member into a second expanded state in which the interior region has a second shape different than the first shape and a volume that is less than a volume of the interior region in the first expanded state.
8. A system for treating an aneurysm comprising any one of the foregoing occlusive devices and an injection shaft configured to be slidably positioned through a proximal end of the occlusive device, wherein the injection shaft is configured to deliver an embolic composition to a location distal of the occlusive device.
9. A device for treating an aneurysm comprising:
a single connector;
an expandable mesh configured to be positioned in a cerebral aneurysm, the mesh forming a bowl when deployed, the bowl having a circumferential distal and a proximal end configured to be positioned over the neck of the aneurysm,
the mesh comprising an outer layer and an inner layer that meet at a fold defining the distal end of the bowl and that are secured together by the connector at the proximal end of the bowl;
in the deployed/expanded state, the mesh defines and surrounds an interior region that is concave towards the aneurysm dome, and wherein the connector is within or distal to the interior region when the device is implanted.
10. A device for treating an aneurysm comprising an expandable mesh configured to be positioned in a cerebral aneurysm, the mesh comprising at least two mesh layers and a membrane positioned between two of the at least two mesh layers.
11. The device of Clause 17, wherein the mesh is a braid.
12. The device of Clause 17, wherein the mesh has a bowl shape.
13. The device of Clause 17, wherein the mesh is fluid impermeable.
14. The device of claim 21, wherein the membrane has a plurality of perforations.

Additional features and advantages of the present technology are described below, and in part will be apparent from the description, or may be learned by practice of the present technology. The advantages of the present technology will be realized and attained by the structure particularly pointed out in the written description and claims hereof as well as the appended drawings.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present disclosure.

FIG. 1A is a partially schematic view of a system for treating an aneurysm in accordance with embodiments of the present technology.

FIG. 1B is an enlarged cross-sectional view of a distal portion of the delivery system shown in FIG. 1A.

FIGS. 2A-2E show an example method of treating an aneurysm with the systems of the present technology.

FIG. 3 is a side view of a neck cover in accordance with embodiments of the present technology.

FIG. 4 is a side view of a neck cover in accordance with embodiments of the present technology.

FIG. 5 is a side view of a neck cover in accordance with embodiments of the present technology.

FIG. 6 is a side view of a neck cover in accordance with embodiments of the present technology.

FIG. 7 is a side view of a neck cover in accordance with embodiments of the present technology.

FIG. 8 is a side view of a neck cover in accordance with embodiments of the present technology.

FIGS. 9A-9D depict use of a neck cover for treating an aneurysm in accordance with embodiments of the present technology.

FIG. 10 is a side view of a neck cover in accordance with embodiments of the present technology.

FIG. 11 is a side view of a neck cover in accordance with embodiments of the present technology.

FIG. 12 is a side view of a neck cover in accordance with embodiments of the present technology.

FIGS. 13A-13C depict use of a neck cover for treating an aneurysm in accordance with embodiments of the present technology.

FIGS. 14A-14C are perspective, side, and cross-sectional views, respectively, of a neck cover in accordance with embodiments of the present technology.

FIGS. 15A-15D depict use of a neck cover for treating an aneurysm in accordance with embodiments of the present technology.

FIGS. 16A and 16B are perspective views of a neck cover in accordance with embodiments of the present technology.

FIG. 17 is a cross-sectional view of a neck cover in accordance with embodiments of the present technology.

FIGS. 18A and 18B are side and bottom views, respectively, of a neck cover in accordance with embodiments of the present technology.

FIG. 19 is a cross-sectional view of a neck cover in accordance with embodiments of the present.

FIGS. 20A and 20B are perspective views of a neck cover in accordance with embodiments of the present technology.

### DETAILED DESCRIPTION

The present technology relates to systems, methods, and devices for treating vascular defects such as aneurysms. In some embodiments, the methods described herein include delivering an embolic composition into the aneurysm sac. The embolic composition can provide a complete or nearly complete volumetric filling of the internal volume of an aneurysm, and/or a complete or nearly complete coverage of the neck of the aneurysm with new endothelial tissue. These features, among others, can lead to a lower recanalization rate than that of platinum coil treatments and faster aneurysm occlusion than that of flow diverters. Additionally, the embolic compositions can be configured to biodegrade over time and thereby have little or no long-term mass effect.

Conventional treatment methods typically use either a low viscosity embolic composition that solidifies (e.g., forms a gel) when exposed to physiological conditions within the aneurysm, or precursor materials that are mixed immediately before delivery to form the final embolic composition. However, the former approach may present challenges with long-term storage stability, while the latter approach introduces additional steps into the treatment procedure and may introduce timing complications (e.g., if the composition gels too quickly, it may clog the delivery catheter; if the composition gels too slowly, it may leak out of the aneurysm). Highly viscous embolic compositions (e.g., embolic compositions having a storage modulus of at least 80 Pa at 37° C. within a linear viscoelastic range of the embolic composition) that are ready for use off-the-shelf without any mixing of precursor materials can address these issues but may be difficult to deliver using conventional systems and devices. For example, conventional delivery systems may not be able to generate and/or withstand the pressures needed to move a highly viscous embolic composition through small-diameter catheters (e.g., microcatheters) used for accessing intracranial aneurysms.

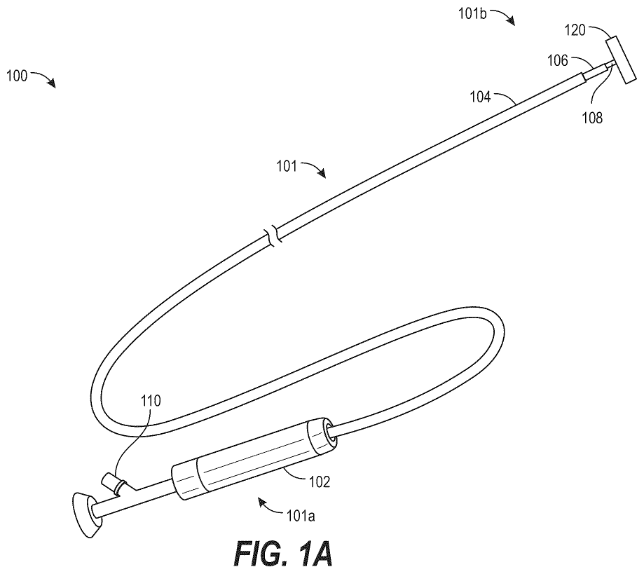

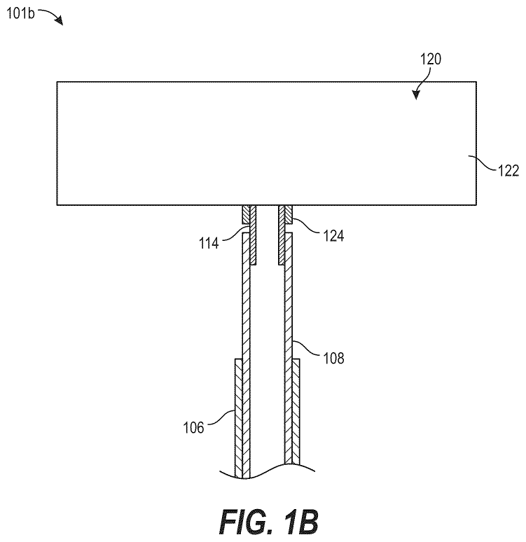

Specific details of devices, systems, and methods for treating aneurysms and/or other vascular defects in accordance with embodiments of the present technology are described herein with reference to FIGS. 1A-20B. Although certain embodiments of these devices, systems, and methods may be described herein primarily or entirely in the context of treating saccular intracranial aneurysms, other contexts are within the scope of the present technology. For example, suitable features of described systems, devices, and methods for treating saccular intracranial aneurysms can be implemented in the context of treating non-saccular intracranial aneurysms, abdominal aortic aneurysms, thoracic aortic aneurysms, renal artery aneurysms, arteriovenous malformations, tumors (e.g. via occlusion of vessel(s) feeding a tumor), perivascular leaks, varicose veins (e.g. via occlusion of one or more truncal veins such as the great saphenous vein), hemorrhoids, and sealing endoleaks adjacent to artificial heart valves, covered stents, and abdominal aortic aneurysm devices, among other examples. Furthermore, it should be understood, in general, that other systems, devices, and methods in addition to those disclosed herein are within the scope of the present disclosure. For example, systems, devices, and methods in accordance with embodiments of the present technology can have different and/or additional configurations, components, procedures, etc. than those disclosed herein. Moreover, systems, devices, and methods in accordance with embodiments of the present disclosure can be without one or more of the configurations, components, procedures, etc. disclosed herein without deviating from the present technology.

The headings provided herein are for convenience only and do not interpret the scope or meaning of the claimed present technology. Embodiments under any one heading may be used in conjunction with embodiments under any other heading.

### I. OVERVIEW OF TREATMENT SYSTEMS OF THE PRESENT TECHNOLOGY

FIG. 1A shows a system 100 for treating aneurysms, such as cerebral aneurysms, according to one or more embodiments of the present technology. As shown in FIG. 1A, the system 100 comprises a delivery system 101 and a neck cover 120. The neck cover 120 (shown schematically) is configured to be detachably coupled to the delivery system 101, and the delivery system 101 is configured to intravascularly position the neck cover 120 within an aneurysm, across the neck of the aneurysm. In some embodiments the system 100 further comprises an embolic kit (not shown). The embolic kit can include an embolic composition and an injector device configured to be fluidly coupled to a proximal portion of the delivery system 101 for injection of the embolic composition into the aneurysm cavity. As detailed below, in some embodiments the embolic composition can be delivered to a space between the neck cover 120 and the dome of the aneurysm to fill and occlude the aneurysm cavity. The neck cover 120 prevents migration of the embolic composition into the parent vessel, and together the neck cover 120 and embolic composition prevent blood from flowing into the aneurysm. Bioabsorption of the embolic composition and endothelialization of the neck cover 120 cause the aneurysm wall to fully degrade, leaving behind a successfully remodeled (aneurysm free) region of the blood vessel.

As shown in FIG. 1A, the delivery system 101 has a proximal portion 101a configured to be extracorporeally positioned during treatment and a distal portion 101b configured to be intravascularly positioned at or within an aneurysm. The delivery system 101 may include a handle 102 at the proximal portion 101a and a plurality of elongated shafts extending between the handle 102 and the distal portion 101b. In some embodiments, for example as shown in FIG. 1A, the delivery system 101 may include a first elongated shaft 104 (such as a guide catheter or balloon guide catheter), a second elongated shaft 106 (such as a microcatheter) configured to be slidably disposed within a lumen of the first elongated shaft 104, and a third elongated shaft 108 configured to be slidably disposed within a lumen of the second elongated shaft 106. The delivery system 101 and/or the third elongated shaft 108 is configured to be detachably coupled at its distal end portion to the neck cover 120 via a connector 124 (see FIG. 1B) of the neck cover 120. In some embodiments, the delivery system 101 does not include the first elongated shaft 104.

The second elongated shaft 106 is generally constructed to track over a conventional guidewire in the cervical anatomy and into the cerebral vessels associated with the brain. The second elongated shaft 106 may also be chosen according to several standard designs that are generally available. For example, the second elongated shaft 106 can have a length that is at least 125 cm long, and more particularly may be between about 125 cm and about 175 cm long. The lumen of the second elongated shaft 106 is configured to slidably receive the neck cover 120 in a radially constrained state. The second elongated shaft 106 can have an inner diameter of about 0.015 inches (0.0381 cm), about 0.017 inches (0.043 cm), about 0.021 inches (0.053 cm), or about 0.027 inches (0.069 cm).

The third elongated shaft 108 can be movable within the first and/or second elongated shafts 104, 106 to position the neck cover 120 at a desired location. The third elongated shaft 108 can be sufficiently flexible to enable manipulation, e.g., advancement and/or retraction, of the neck cover 120 through tortuous passages. Tortuous passages can include, for example, catheter lumens, microcatheter lumens, blood vessels, urinary tracts, biliary tracts, and airways. The third elongated shaft 108 can be formed of any material and in any dimensions suitable for the task(s) for which the system 100 is to be employed. In some embodiments, at least the distal portion of the third elongated shaft 108 can comprise a flexible metal hypotube. The hypotube, for example, can be laser cut along all or a portion of its length to impart increased flexibility. In some embodiments, the third elongated shaft 108 can be surrounded over some or all of its length by a lubricious coating, such as polytetrafluoroethylene (PTFE).

When used, the embolic composition may be pre-loaded into the injector, or at least some of the embolic composition may be provided separately. The embolic composition can be any material suitable for forming a solid or semi-solid structure (e.g., a hydrogel) that partially or completely occludes the interior cavity of the aneurysm. For example, the embolic composition can include one or more polymers, such as a synthetic polymer (e.g., poly(glycolide), poly(lactide), poly(vinyl alcohol)), a biopolymer (e.g., chitosan, gelatin, silk, cellulose, alginate, hyaluronic acid), or a combination thereof. The embolic composition can optionally include one or more components to facilitate gelation and/or enhance storage stability, such as cross-linking agents, stabilizers, thickeners, spacers, etc. Optionally, the embolic composition can include a contrast agent to enable visualization (e.g., iohexol, iopromide, ioversol, iopamidol, iodixanol, ioxilan, iothalamate/meglumine, ioxaglate/meglumine, diatrizoate/meglumine). The embolic composition can be biodegradable or non-biodegradable.

The embolic composition can be provided in many different formats. In some embodiments, for example, the embolic composition comprises two or more precursor materials that are mixed prior to or during delivery to the aneurysm. Upon mixing, the precursor materials can chemically react and/or physically interact to form a gel or other solid or semi-solid structure for occluding the aneurysm. Alternatively, the embolic composition can be a preformed composition that is ready for use without any mixing of precursor materials. In such embodiments, the embolic composition can be a highly viscous material that is sufficiently solid to fill and occlude the aneurysm without requiring further chemical reactions and/or physical interactions.

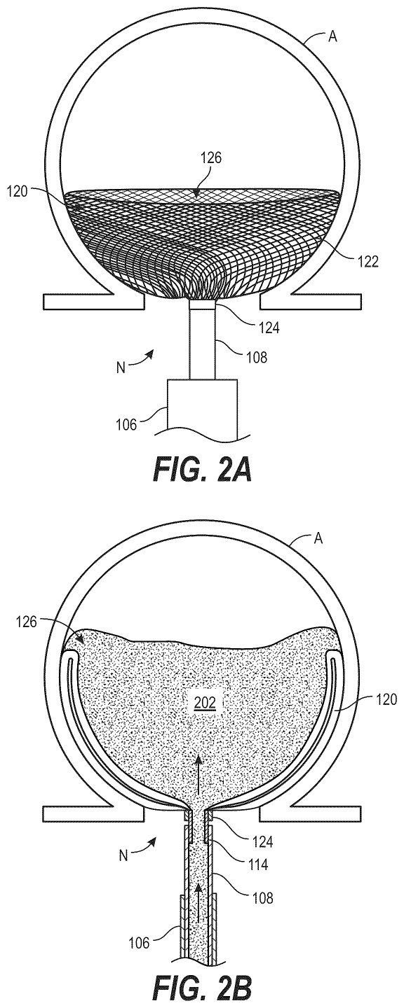

The system 100 can further include a conduit configured to guide the embolic composition to a space between at least a portion of the neck cover 120 and the aneurysm dome. In some embodiments, the conduit is incorporated into the delivery system 101. For example, as depicted in the enlarged cross-sectional view of the distal portion 101b shown in FIG. 1B, the conduit can comprise a combination of the third elongated shaft 108 and an extension 114 fixed to a distal end portion of the third elongated shaft 108. The extension 114 can be a tubular member that extends distally from the third elongated shaft 108, through the connector 124, and through the neck cover 120, at least when the neck cover 120 is in an expanded state. When the neck cover 120 is collapsed within the lumen of the third elongated shaft 108 during delivery, a portion of the neck cover 120 may extend distally of the extension 114. The length of the extension 114 can be such that, when the distal portion 101b of the delivery system 101 is positioned at the aneurysm with the neck cover 120 in an expanded state (for example, as shown in FIG. 2A), a distal terminus of the extension 114 is even with the distal end of the connector 124, distal of the connector 124 but proximal of a distal end of the neck cover 120, or even with or distal of the distal end of the neck cover 120. It may be beneficial for the extension 114 to be as short as possible to ensure the extension 114 remains sufficiently spaced apart from the fragile aneurysm wall.

In some embodiments, the extension 114 comprises an atraumatic member, such as a soft, flexible coil. In other embodiments, the extension 114 comprises a flexible tube having a continuous sidewall (i.e., not formed of a coiled member). In any case, a distal end portion of the injector 204 can be fluidly coupled to a proximal end portion of the third elongated shaft 108 via a port 110. The port 110 can be located at the proximal portion 101a of the delivery system 101, such as on or proximal to the handle 102. The pressure generated at the injector 204 can cause the embolic composition 202 to flow through the lumen of the third elongated shaft 108, through the lumen of the extension 114, and into the aneurysm cavity. Once the embolic composition 202 has sufficiently filled the aneurysm cavity, the neck cover 120 and extension 114 can be detached via electrolytic detachment that severs a region of the extension 114 exposed between the third elongated shaft 108 and the neck cover 120.

According to several embodiments, the conduit may comprise an additional elongated shaft (not shown). The additional elongated shaft can be delivered to the aneurysm through one or more of the first, second, and/or third elongated shafts 104, 106, 108, or may be delivered separately (i.e., outside of) the delivery system 101. In such embodiments, a proximal end portion of the elongated shaft is configured to be fluidly coupled to the injector 204 via the port 110. Methods for delivering the embolic composition 202 through a separate elongated shaft are discussed below.

The neck cover 120 may comprise an expandable element having a low-profile or constrained state while positioned within a catheter (such as the second elongated shaft 106) for delivery to the aneurysm and an expanded, deployed state for positioning within the aneurysm. In some embodiments the neck cover 120 comprises a mesh 122 (shown schematically in FIG. 1B) and a connector 124 coupled to the mesh 122. The connector 124 is configured to be coupled to one or more components of the delivery system 101, such as the third elongated shaft 108 and/or extension 114. The mesh 122 can be formed of a resilient material and shape set such that upon exiting the second elongated shaft 106, the mesh 122 self-expands to a predetermined shape. The mesh 122 can have any shape or size in the expanded state that enables the mesh 122 to cover the aneurysm neck. In some embodiments, for example as shown in FIG. 2A, the mesh 122 can be configured to assume a bowl shape. Other shapes are possible. The mesh 122 has a porosity sufficient to prevent leakage of the embolic composition 202 into the parent vessel.

In some embodiments, the mesh 122 is formed of a plurality of braided filaments that have been heat-set to assume a predetermined shape when released from the constraints of the delivery catheter. The mesh 122 may be formed of metal wires, polymer wires, or both, and the wires may have shape memory and/or superelastic properties. The mesh 122 may be formed of 24, 32, 36, 48, 64, 72, 96, 128, or 144 filaments. The mesh 122 may be formed of a range of filament or wire sizes, such as wires having a diameter of from about 0.0004 inches to about 0.0020 inches, or of from about 0.0009 inches to about 0.0012 inches. In some embodiments, each of the wires or filaments have a diameter of about 0.0004 inches, about 0.0005 inches, about 0.0006 inches, about 0.0007 inches, about 0.0008 inches, about 0.0009 inches, about 0.001 inches, about 0.0011 inches, about 0.0012 inches, about 0.0013 inches, about 0.0014 inches, about 0.0015 inches, about 0.0016 inches, about 0.0017 inches, about 0.0018 inches, about 0.0019 inches, or about 0.0020 inches. In some embodiments, all of the filaments of the braided mesh 122 may have the same diameter. For example, in some embodiments, all of the filaments have a diameter of no more than 0.001 inches. In some embodiments, some of the filaments may have different cross-sectional diameters. For example, some of the filaments may have a slightly thicker diameter to impart additional strength to the braid. In some embodiments, some of the filaments can have a diameter of no more than 0.001 inches, and some of the filaments can have a diameter of greater than 0.001 inches. The thicker filaments may impart greater strength to the braid without significantly increasing the device delivery profile, with the thinner wires offering some strength while filling out the braid matrix density.

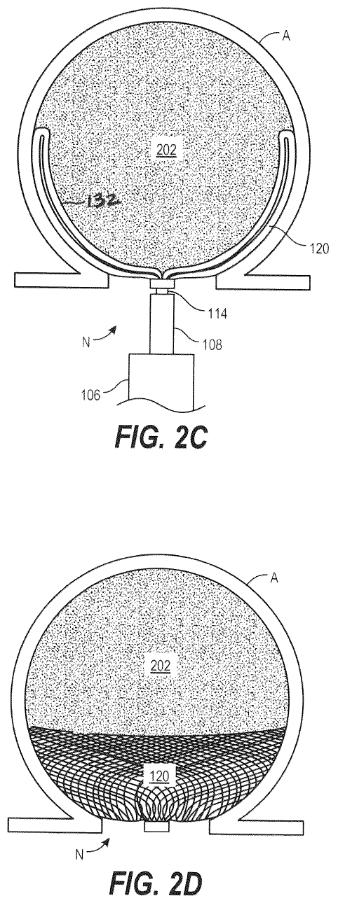

In some embodiments, the mesh 122 can be a non-braided structure, such as a laser-cut stent. Moreover, while the mesh 122 shown in FIGS. 2A-2D is a dual-layer mesh, in some embodiments the mesh 122 may comprise more or fewer layers (e.g., a single layer, three layers, four layers, etc.). Additional examples of mesh structures for use as neck covers of the present technology are shown and described below with reference to FIGS. 3-20B.

### II. SELECTED METHODS FOR TREATING ANEURYSMS WITH THE SYSTEMS OF THE PRESENT TECHNOLOGY

A physician may begin by intravascularly advancing the second elongated shaft 106 towards an intracranial aneurysm A with the neck cover 120 in a low-profile, collapsed state and coupled to a distal end portion of the third elongated shaft 108. A distal portion of the second elongated shaft 106 may be advanced through a neck N of the aneurysm A to locate a distal opening of the second elongated shaft 106 within an interior cavity of the aneurysm A. The third elongated shaft 108 may be advanced distally relative to the second elongated shaft 106 to push the neck cover 120 through the opening at the distal end of the second elongated shaft 106, thereby releasing the neck cover 120 from the shaft 108 and enabling the neck cover 120 to self-expand into an expanded, deployed state.

FIG. 2A shows the neck cover 120 in an expanded, deployed state, positioned in an aneurysm cavity and still coupled to the third elongated shaft 108. In the expanded, deployed state, all or a portion of the neck cover 120 may generally conform to the curved inner surface of the aneurysm A. As detailed below with reference to FIGS. 3-21B, in some embodiments the neck cover 120 is biased towards a predetermined shape that is concave towards the aneurysm dome and defines a cavity 126. In several embodiments the neck cover 120 includes a broad distal surface that effectively compartmentalizes the aneurysm space into a first region largely defined by the walls of the neck cover and a second region between the distal surface of the neck cover and the aneurysm dome.

As illustrated in FIG. 2B, an embolic composition 202 (such as any embolic composition disclosed herein) can be injected through the third elongated shaft 108 and extension 114 to a space between the neck cover 120 and an inner surface of the aneurysm wall. In other embodiments, the embolic composition 202 can be delivered through another elongated shaft (not shown) separate from the third elongated shaft 108 and extension 114. As additional embolic composition 202 is delivered, it fills the cavity 126 and all or a portion of the volume of the aneurysm cavity. It is beneficial to fill as much space in the aneurysm as possible, as leaving voids within the aneurysm sac may cause delayed healing and increased risk of aneurysm recanalization and/or rupture. While the scaffolding provided by the neck cover 120 across the neck helps thrombosis of blood form in any gaps and healing at the neck N, the substantial filling of the cavity prevents rupture acutely and does not rely on the neck cover 120. In some embodiments, the embolic composition 202 may fill greater than 40%, 50%, 60%, 65%, 70%, 75%, 80%, 85%, or 90% of the aneurysm sac volume.

FIG. 2C is a cross-sectional view of the neck cover 120 still attached to the delivery system just after completion of delivery of the embolic composition 202. During and after delivery, the embolic composition 202 exerts a substantially uniform downward pressure (i.e., towards the parent vessel) on the neck cover 120 that further seals and stabilizes the neck cover 120 around the neck N of the aneurysm A. Moreover, the embolic composition 202 along the distal wall 132 provides additional occlusion. In some embodiments, the embolic composition 202 completely or substantially completely occludes the pores of the adjacent layer or wall of the neck cover 120 such that blood cannot flow past the embolic composition 202 into the aneurysm cavity. It is desirable to occlude as much of the aneurysm as possible, as leaving voids of gaps can enable blood to flow in and/or pool, which may continue to stretch out the walls of aneurysm A. Dilation of the aneurysm A can lead to recanalization and/or herniation of the neck cover 120 and/or embolic composition 202 into the parent vessel and/or may cause the aneurysm A to rupture. Both conditions can be fatal to the patient.

As shown in FIG. 2D, once delivery of the embolic composition 202 is complete, the delivery system 101 and/or third elongated shaft 108 can be detached from the neck cover 120 (electrolytically or mechanically) and withdrawn from the patient's body. In those embodiments comprising a separate elongated shaft for delivering the embolic composition 202, the elongated shaft can be withdrawn before, during, or after detachment of the third elongated shaft 108 from the neck cover 120.

Over time natural vascular remodeling mechanisms and/or bioabsorption of the embolic composition 202 may lead to formation of a thrombus and/or conversion of entrapped thrombus to fibrous tissue within the internal volume of the aneurysm A. These mechanisms also may lead to cell death at a wall of the aneurysm and growth of new endothelial cells between and over the filaments of the neck cover 120. Eventually, the thrombus and the cells at the wall of the aneurysm may fully degrade, leaving behind a successfully remodeled region of the blood vessel.

In some embodiments, contrast agent can be delivered during advancement of the neck cover 120 and/or embolic composition 202 in the vasculature, deployment of the neck cover 120 and/or embolic composition 202 at the aneurysm A, and/or after deployment of the neck cover 120 and/or embolic composition 202 prior to initiation of withdrawal of the delivery system. The contrast agent can be delivered through the second elongated shaft 106, the conduit, or through another catheter or device commonly used to deliver contrast agent. The aneurysm (and devices therein) may be imaged before, during, and/or after injection of the contrast agent, and the images may be compared to confirm a degree of occlusion of the aneurysm.

As shown in FIG. 2E, in some embodiments, the system 100 may comprise two separate elongated shafts (e.g., microcatheters), with one elongated shaft dedicated to delivery of the embolic composition 202 (e.g., a fourth elongated shaft 128), and the other elongated shaft dedicated to the delivery of the neck cover 120 (e.g., the third elongated shaft 108). In such embodiments, the fourth elongated shaft 128 can be fluidly coupled to the injector device 204 to form at least part of the conduit for conveying the embolic composition 202 into the aneurysm A. The fourth elongated shaft 128 may be intravascularly advanced to the aneurysm A and through the neck N such that that a distal tip of the fourth elongated shaft 128 is positioned within the aneurysm cavity. In some embodiments, the fourth elongated shaft 128 may be positioned within the aneurysm cavity such that the distal tip of the shaft 128 is near the dome of the aneurysm A.

The third elongated shaft 108 containing the neck cover 120 may be intravascularly advanced to the aneurysm A and positioned within the aneurysm cavity adjacent the fourth elongated shaft 128. The neck cover 120 may then be deployed within the aneurysm sac. As the neck cover 120 is deployed, it pushes the fourth elongated shaft 128 outwardly towards the side of the aneurysm A, and when fully deployed the neck cover 120 holds or “jails” the fourth elongated shaft 128 between an outer surface of the neck cover 120 and the inner surface of the aneurysm wall.

The embolic composition 202 may then be delivered through the fourth elongated shaft 128 to a position between the inner surface of the aneurysm wall and the outer surface of the neck cover 120. For this reason, it may be beneficial to initially position the distal tip of the fourth elongated shaft 128 near the dome (or more distal surface) of the aneurysm wall. This way, the “jailed” fourth elongated shaft 128 will be secured by the neck cover 120 such that the embolic composition 202 gradually fills the open space in the aneurysm sac between the dome and the neck cover 120.

### III. SELECTED EMBODIMENTS OF NECK COVERS

Treatment of an aneurysm in accordance with the present technology can comprise positioning an occlusive element over a neck of the aneurysm (e.g., a neck cover) and/or positioning an embolic composition between the neck cover and the wall of the aneurysm to fill at least a portion of the cavity of the aneurysm. A neck cover can be configured to prevent or limit blood flow across the neck cover and into the aneurysm. For example, a neck cover can have a sufficiently low porosity to provide sufficient occlusion. In some embodiments, the neck cover can comprise two or more layers such that a combined porosity of the neck cover is smaller than an individual porosity of each individual layer. In some cases, it may be advantageous to occlude as much of the aneurysm as possible, as blood may flow into and/or pool within voids remaining within the aneurysm cavity, which may in turn stretch the walls of aneurysm and lead to serious, potentially-fatal conditions. Thus, various aspects of the present technology are directed to neck covers configured to be used in conjunction with an embolic composition configured to fill a space between a wall of an aneurysm and a neck cover. Such neck covers can be configured to support an embolic composition within an aneurysm and prevent herniation of the embolic composition into the adjacent vessels. In some embodiments, a neck cover can be configured to provide visual feedback to an operator regarding a position of the neck cover within the aneurysm and/or a degree to which the aneurysm cavity has been filled with an embolic composition. Several of such embodiments, for example, are described below with respect to FIGS. 3-20B.

FIG. 3 is a side view of a neck cover 300 in accordance with several embodiments of the present technology. The neck cover 300 may comprise a mesh having a proximal portion 300a configured to be positioned over a neck of an aneurysm and a distal portion 300b configured to be positioned within a cavity of the aneurysm. The neck cover 300 can be configured to prevent or limit blood flow into the aneurysm cavity and/or support an embolic composition delivered to the aneurysm cavity. In some embodiments, the neck cover 300 is configured to provide visual feedback to a clinician delivering the neck cover 300 and/or an embolic composition. The mesh may be biased towards a predetermined shape when the mesh is in an expanded, unconstrained state.

In some embodiments, the neck cover 300 comprises a plurality of braided or woven filaments that are secured to one another at the proximal portion of the neck cover 300 via a securing means (not shown in FIG. 3). The securing means can be, for example, a mechanical restraint (e.g., one or more bands, etc.) or incorporation of a cured material. Details regarding the use of cured materials to secure the filaments and/or neck covers of the present technology are disclosed in U.S. patent application Ser. No. 17/816,380, filed Jul. 29, 2022, titled DEVICES, SYSTEMS, AND METHODS FOR TREATING ANEURYSMS, and U.S. patent application Ser. No. 17/816,398, filed Jul. 30, 2022, titled, both of which are incorporated by reference herein in their entireties.

As shown in FIG. 3, the mesh can comprise a wall surrounding an interior region 306 and comprising a first layer 308, a second layer 310, and an annular rim 312 where the first layer 308 is continuous with the second layer 310 at the distal portion 300b of the cover 300. Each of the first layer 308 and the second layer 310 of the wall can extend between the rim 312 and the gathered proximal end regions. When the neck cover 300 is in an expanded state, for example as shown in FIG. 3, the wall of the mesh can comprise a first region 314 in which the first layer 308 and the second layer 310 substantially conform to one another and a second region 316 in which the first layer 308 and the second layer 310 are spaced apart from one another. In various embodiments, the first layer 308 and the second layer 310 can be spaced apart at the second region 316 by a distance d. The distance d may be generally constant or may vary at different radial and/or circumferential positions.

In an expanded state, the second portion 310 of the neck cover 300 bows inwardly from the third portion 312 towards the interior region 306 to form a cavity 318 at the distal portion 300b of the neck cover 300. The cavity 318, for example, can be bound by the second portion 310 of the wall and a plane lying on the third portion 312. In some embodiments, the wall of the mesh forms a bowl shape defining a substantially cylindrical cavity 318. In some embodiments, the neck cover 300 and/or mesh includes a recessed portion 320 at the proximal portion 300a. The recessed portion 320 can surround all or a portion of the proximal coupler. In some embodiments, the neck cover 300 and/or mesh does not include a recessed portion 320 at the proximal portion 300a.

Because the second portion 310 bows inwardly, the neck cover 300 is less likely to elongate when deployed in the aneurysm and/or elongates less (as compared to neck covers with an outward bowing portion). In addition, the inwardly bowed second portion 310 has a shape and curvature corresponding to a desired shape and curvature of the second portion 310 in a semi-collapsed state (for example with reference to FIGS. 2B and 2C). Thus to assume the semi-collapsed state, the second portion 310 does not have to invert in response to forces applied by an embolic composition delivered to the aneurysm. Such lack of inversion may be desirable because inversion of the neck cover 300 may require higher delivery force and/or provide for less consistent delivery of the neck cover 300. In place of inversion, the second portion 310 of the neck cover 300 can simply displace closer to the first portion 308 (e.g., such that the distance d is decreased) in response to forces applied to the neck cover 300 during and/or after delivery of an embolic composition to the neck cover 300 and/or aneurysm.

In some embodiments, for example as shown in FIG. 3, the neck cover 300 and/or mesh is formed of a plurality of braided filaments 302, each having first and second ends and a length measured therebetween. The first and second ends of the filaments 302 of the neck cover 300 are secured relative to one another at the same location (the proximal coupler, not shown). The proximal coupler is configured to be attached to the first and second ends at the distal-most region where the first and second ends come together. As shown in FIG. 3, the second ends come together at the recessed portion 320 and form a proximally-extending column that extends into the collected first ends. The resulting mesh structure thus has a “double layer” delivery configuration in which the first and second portions 308, 310 of the wall radially overlap one another when the neck cover 300 is in a low-profile state and contained within a delivery catheter. The dual layer delivery configuration of the neck cover 300 advantageously allows for a mesh to have a lower porosity than a porosity of either of the individual layers, which can provide greater occlusion of the aneurysm as compared to neck covers having only a single layer.

The mesh of the neck cover 300 may be formed of metal wires, polymer wires, or both, and the wires may comprise a resilient material and/or a material having shape memory and/or superelastic properties. The mesh may be formed of 24, 32, 36, 48, 64, 72, 96, 128, or 144 filaments. The mesh may be formed of a range of filament or wire sizes, such as wires having a diameter of from about 0.0004 inches to about 0.0020 inches, or of from about 0.0009 inches to about 0.0012 inches. In some embodiments, each of the wires or filaments have a diameter of about 0.0004 inches, about 0.0005 inches, about 0.0006 inches, about 0.0007 inches, about 0.0008 inches, about 0.0009 inches, about 0.001 inches, about 0.0011 inches, about 0.0012 inches, about 0.0013 inches, about 0.0014 inches, about 0.0015 inches, about 0.0016 inches, about 0.0017 inches, about 0.0018 inches, about 0.0019 inches, or about 0.0020 inches. In some embodiments, all of the filaments of the braided mesh may have the same diameter. For example, in some embodiments, all of the filaments have a diameter of about 0.001 inches. In some embodiments, some of the filaments may have different cross-sectional diameters. For example, some of the filaments may have a slightly thicker diameter to impart additional strength to the braided layers. In some embodiments, some of the filaments can have a diameter of about 0.001 inches, and some of the filaments can have a diameter of greater than 0.001 inches. The thicker filaments may impart greater strength to the braid without significantly increasing the device delivery profile, with the thinner wires offering some strength while filling-out the braid matrix density.

It may be useful for a neck cover of the present technology to be visible to a clinician (e.g., via fluoroscopy, etc.) during delivery and/or expansion of the neck cover and/or delivery of an embolic composition. For example, deformation of the neck cover from an expanded state to a semi-collapsed state in response to delivery of an embolic composition can be visualized to assess the degree of filling of the aneurysm and/or coverage of the neck. Thus, while inversion of the neck cover may be less desirable, it may be useful for a neck cover to be configured to substantially deform in response to delivery of an embolic composition to provide visual feedback earlier and/or at a higher resolution.

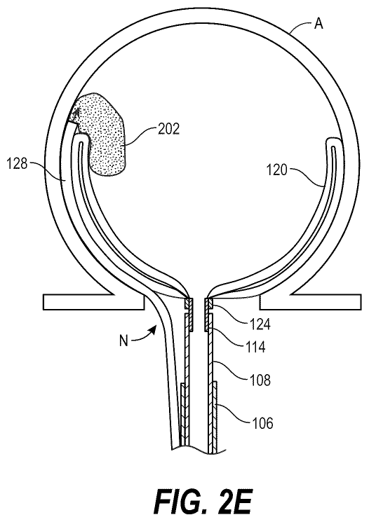

FIGS. 4-20B illustrate representative examples of neck covers 400-2100 with various geometries configured in accordance with embodiments of the present technology. Any of the neck covers disclosed herein, including the neck covers shown in FIGS. 3-20B, can be used with the system 100 of the present technology. In many cases, the features of the neck covers of FIGS. 3-20B can be generally similar to the features of neck cover 300 of FIG. 3. Accordingly, like numbers (e.g., first layer 308 versus first layer 408) are used to identify similar or identical components in FIGS. 3-20B, and the discussion of the neck covers in FIGS. 4-20B will be limited to those features that differ from the neck cover 300 of FIG. 3. Additionally, any of the features of the neck covers of FIGS. 4-20B can be combined with each other and/or with the features of the neck cover 300 of FIG. 3.

FIG. 4 is a side view of an example neck cover 400 in accordance with the present technology. The neck cover 400 may comprise a mesh having a proximal portion 400a configured to be positioned over a neck of the aneurysm, a distal portion 400b configured to be positioned within the aneurysm, and a longitudinal axis L extending between the proximal and distal portions 400a, 400b. The mesh can have an outer layer 408 and an inner layer 410 that meet distally at an intermediate region 409 at the distal end portion 400b of the mesh. The inner and outer layers 410, 408 can meet proximally at the proximal end portion 400a of the mesh via a securing means 401. As previously mentioned, the securing means 401 can comprise a mechanical restraint, a cured material, or other suitable means.

When the mesh is in an expanded, unconstrained state, an inner surface 408a of the outer layer 408 and an inner surface 410a of the inner layer 410 can be separated by a radial distance d. The inner surfaces 408a, 410a of the outer and inner layers 408, 410 along with the inner surface of the intermediate region 409 together enclose a first cavity 406. An outer surface 410b of the inner layer 410 can define a second cavity that opens distally at a distal opening 414 of the device. The inner surface 410a of the inner layer 408 can have a first region 430 extending from the opening 414 and/or intermediate region 409 proximally. The first region 430 can be concave towards the inner surface 408a of the outer layer 408. The inner surface 410a of the inner layer 410 can comprise a second region 432 that extends proximally from the first region 430, and the second region is convex towards the inner surface of the outer layer (or concave towards the second cavity). It can be beneficial to use braids having preferential bend points when supplementing embolic composition delivery, as treatment time and success depends in large part on how the mesh and embolic composition react. In some embodiments, the outer layer defines a diameter that increases then decreases along the longitudinal axis L of the mesh between the proximal and distal end portions. Said another way, the outer layer can define a first diameter at the proximal end portion of the mesh, a second diameter at an intermediate portion of the mesh, and a third diameter at the distal end portion of the mesh, wherein the individual first and third diameters are less than the second diameter.

Methods for treating an aneurysm in accordance with the present technology can include positioning a distal end of an elongated shaft in an aneurysm cavity and releasing an occlusive member from the elongated shaft into the aneurysm cavity. The occlusive member can comprise outer and inner layers. Releasing the occlusive member can allow the occlusive member to self-expand into a first expanded state in which inner surfaces of the inner and outer layers enclose an interior region having a first shape and an outer surface of the inner layer defines a cavity. The method further includes delivering an embolic element into the cavity. As the embolic element continues to be delivered, the embolic element applies a radially outward force to the outer surface of the inner layer, thereby causing the inner layer to move radially outward towards the outer layer and transforming the occlusive member into a second expanded state in which the interior region has a second shape different than the first shape and a volume that is less than a volume of the interior region in the first expanded state.

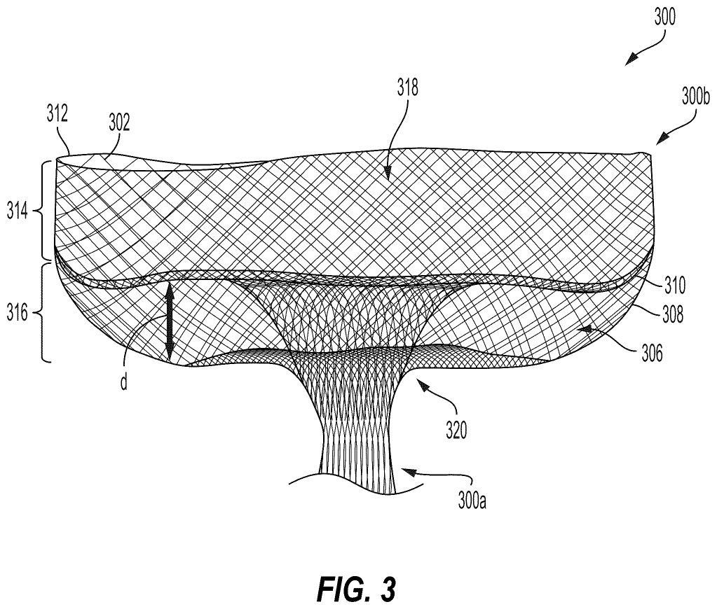

FIG. 5 shows a neck cover similar to that shown in FIG. 4, except in FIG. 5 the outer diameter of the braid mostly decreases from the intermediate portion down to the proximal end portion of the cover 500.

In any of the embodiments disclosed herein, the inner layer and/or outer layer can include baffles.

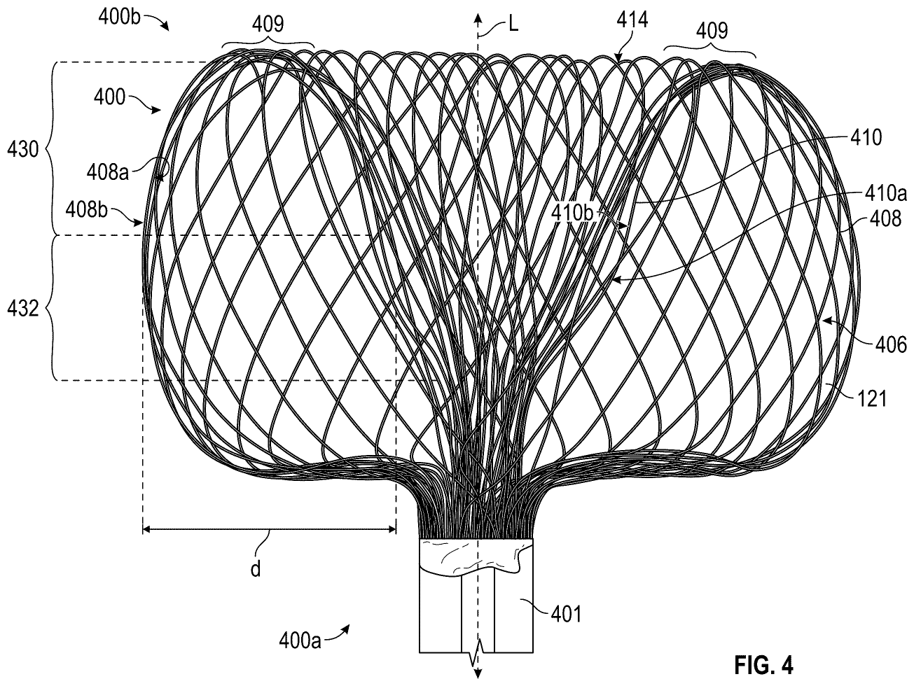

FIG. 6 depicts a neck cover 1000 in accordance with several embodiments of the present technology. The neck cover 1000 may comprise a mesh having a proximal portion 1000a configured to be positioned over a neck of the aneurysm and a distal portion 1000b. In some embodiments, the neck cover 1000 comprises a proximal coupler 1004. The mesh may be biased towards a predetermined shape when the mesh is in an expanded, unconstrained state. The mesh can comprise a wall surrounding an interior region 1006 and comprising a first portion 1008, a second portion 1010, and a third portion 1012. The first portion 1008 and the second portion 1010 of the wall can each extend between the third portion 1012 and the proximal coupler.

In an expanded state, an outer profile of the neck cover 1000 defined by the first portion 1008 can have an oblate spheroidal shape. For example, as shown in FIG. 6, the first portion 1008 can comprise a first region 1008a extending substantially laterally away from a proximal coupler and a second region 1008b extending distally, and in some cases laterally, from the first region 1008a to the third portion 1012. The neck cover 1000 and/or mesh can be substantially flat at the proximal portion 1000a (e.g., the first region 1008a of the first portion 1008 of the wall) and/or may comprise a recessed portion at the proximal portion 1000a. The recessed portion can surround all or a portion of the proximal coupler. In some embodiments, the neck cover 1000 and/or mesh does not include a recessed portion at the proximal portion 1000a.

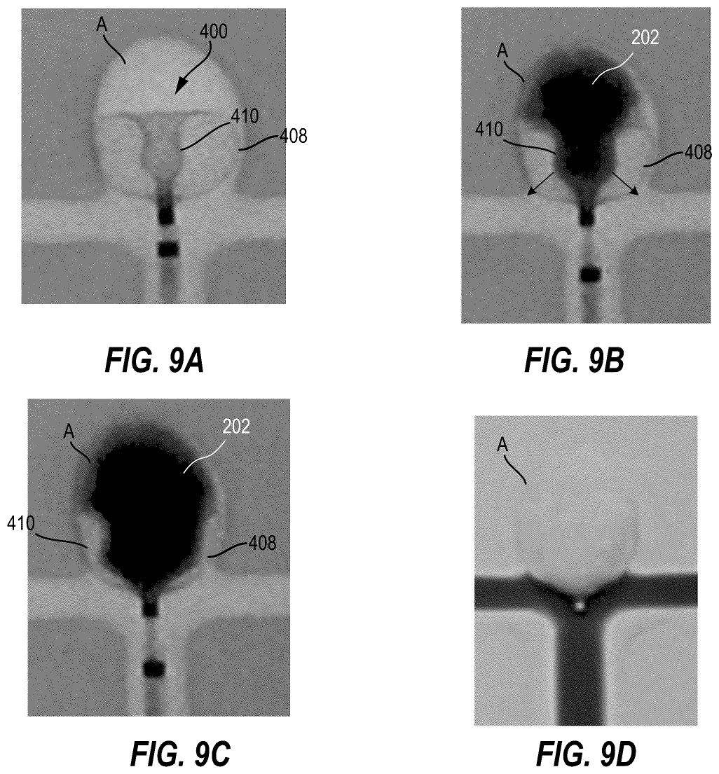

The second portion 1010 of the neck cover 1000 can bow inwardly from the third portion 1012 towards the interior region 1006 to define a cavity 1014 of the neck cover 1000. The cavity 1014 can, for example, be bound by the second portion 1010 of the wall and a plane lying tangent to a peak of the third portion 1012. In some embodiments, a parameter of the second portion 1010 can be configured to facilitate deformation of the second portion 1010 in a specific manner in response to delivery of an embolic composition. For example, a curvature of the second portion 1010 can vary along a length of the second portion 1010 (e.g., the second portion 1010 can undulate along its length) to facilitate movement of the second portion 1010 towards the first portion 1008 when an embolic composition is delivered to the cavity 1014 and/or aneurysm. In some embodiments, the second portion 1010 can comprise a first region 1010a that is concave to the cavity 1014 and a second region 1010b that is convex to the cavity 1014. Although FIG. 10 depicts the second portion 1010 undulating between one concave region and one convex region, the second portion 1010 can undulate between any suitable number of concave and convex regions.

The curvature of the second portion 1010 and/or undulations of the second portion 1010 (e.g., changes in concavity and/or convexity) can facilitate deformation of the second portion 1010 in a specific manner in response to forces applied to the second portion 1010 by an embolic composition. For example, the first portion 1008 of the neck cover 1000 shown in FIG. 10 is concave to the cavity 1014. Thus, for the second portion 1010 to conform to the first portion 1008, the second portion 1010 must be or become concave to the cavity 1014. If the second portion 1010 is convex to the cavity 1014 in the expanded state, the second portion 1010 must invert to change its curvature and conform to the concave first portion 1008. Such inversion may be difficult to achieve accurately and/or precisely in vivo. In contrast, the first region 1010a of the second portion 1010 shown in FIG. 6 is concave to the cavity 1014 and, rather than inverting, will expand radially outwardly to conform to the first portion 1008 during and/or after delivery of an embolic composition. In some embodiments, the second region 1010b being convex to the cavity 1014 can facilitate collection of the second portion 1010 at the proximal coupler 1004.

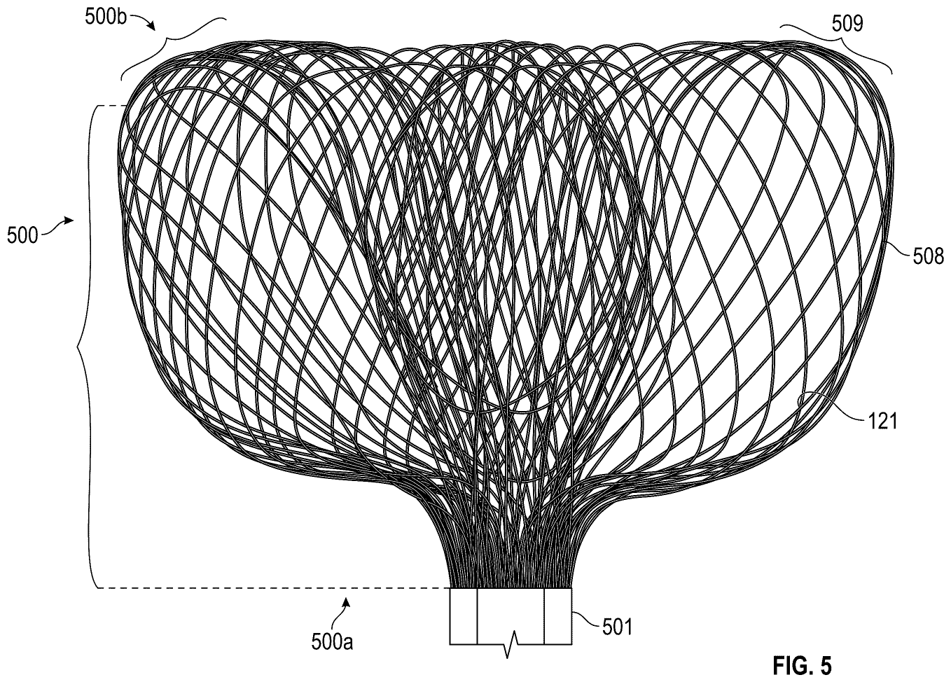

In some embodiments, a parameter of the third portion 1012 of can be selected based on a desired deformation of the neck cover 1000 in response to delivery of an embolic composition to the neck cover 1000 and/or aneurysm. For example, the ends of the third portion 1012 shown in FIG. 6 are radially spaced apart and the third portion 1012 follows an arcuate path between its ends. Such configuration of the third portion 1012 can reduce a force required to deform the neck cover 1000 with an embolic composition, improve a precision of deformation of the neck cover 1000, etc. A spacing of the ends of the third portion 1012 can vary based on a desired performance of the neck cover 1000. For example, FIG. 7 depicts a neck cover 1100 having a third portion 1112 with ends spaced apart by a greater distance (e.g., wider) than third portion 1012. The neck cover 1100 can be similar to any of the neck covers disclosed herein, except as detailed below. The neck cover 1100 can comprise a mesh having a proximal portion 1100a configured to be positioned over a neck of the aneurysm and a distal portion 1100b. In some embodiments, the neck cover 1100 comprises a proximal coupler 1104. The mesh may be biased towards a predetermined shape when the mesh is in an expanded, unconstrained state. The mesh can comprise a wall surrounding an interior region 1106 and comprising a first portion 1108, a second portion 1110, and the third portion 1112. The first portion 1108 and the second portion 1110 of the wall can each extend between the third portion 1112 and the proximal coupler. The second portion 1110 can at least partially define a cavity 1114. In some embodiments, the first portion 1108 extends laterally and distally from the proximal coupler 1104 to the third portion 1112 such that a radially widest portion of the neck cover 1100 is at or near a distal portion 1100b of the neck cover 1100. The third portion 1112 can have a large radius of curvature such that distal ends of the first and second portions 1108, 1110 are spaced apart. During use, an embolic composition can be delivered through the cavity 1114 and into to the aneurysm cavity to apply radially outward and compressive forces to the second portion 1110 of the wall and/or the third portion 1112 such that the second portion 1110 moves closer to the first portion 1108. The third portion 1112 can have a shape configured to facilitate such motion and/or deformation of the second portion 1110.

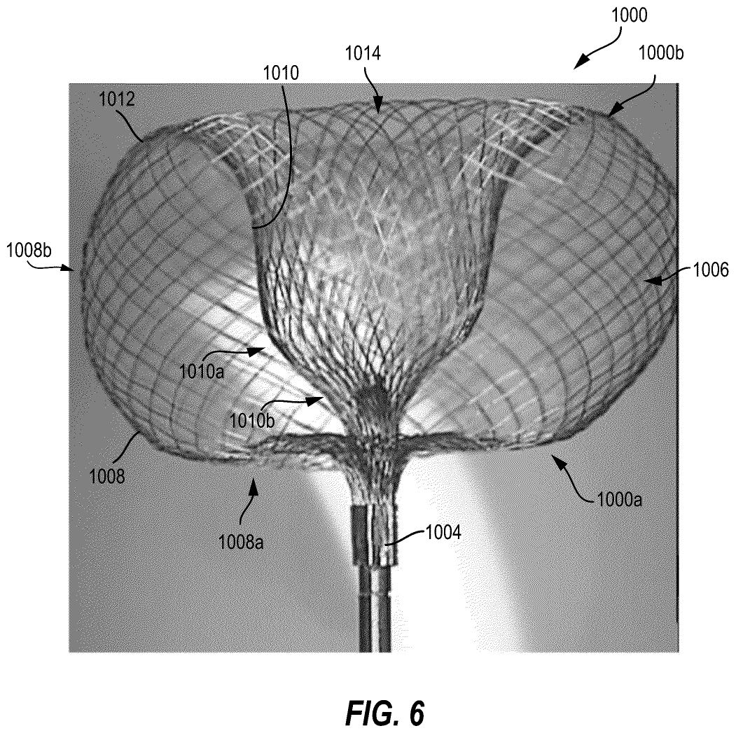

FIG. 8 depicts a neck cover 1200 in accordance with several embodiments of the present technology. The neck cover 1200 can be similar to any of the neck covers disclosed herein, except as detailed below. The neck cover 1200 can comprise a mesh having a proximal portion 1200a configured to be positioned over a neck of the aneurysm and a distal portion 1200b. In some embodiments, the neck cover 1200 comprises a proximal coupler 1204. The mesh may be biased towards a predetermined shape when the mesh is in an expanded, unconstrained state. The mesh can comprise a wall surrounding an interior region 1206 and comprising a first portion 1208, a second portion 1210, and a third portion 1212. The first portion 1208 and the second portion 1210 of the wall can each extend between the third portion 1212 and the proximal coupler 1204. The second portion 1210 can at least partially define a cavity 1214. In some embodiments, the first portion 1208 comprises a first region 1208a extending laterally away from the proximal coupler 1204 and a second region 1208b extending distally from the first region 1208a to the third portion 1212. In these and other embodiments, an outer profile of the neck cover 1200 defined by the first portion 1208 can be substantially barrel shaped. The third portion 1212 can extend between the first portion 1208 and the second portion 1210 along an arcuate path, which can facilitate deformation of the second portion 1210 during filling of the aneurysm. As show in FIG. 8, ends of the third portion 1212 can be spaced apart to a lesser degree than as shown in FIGS. 6 and 7, for example. In some embodiments, the second portion 1210 can comprise a first region 1210a that is concave to the cavity 1214, a second region 1210b that is convex to the cavity 1214, and/or a third region 1210c that radially converges and extends proximally from the second region 1210b to the proximal coupler 1204. As an embolic composition travels distally through the cavity 1214, outward pressure applied to the second portion 1210 by the embolic composition can cause the third region 1210c to expand before expansion of the second region 1210b and/or the first region 1210a.

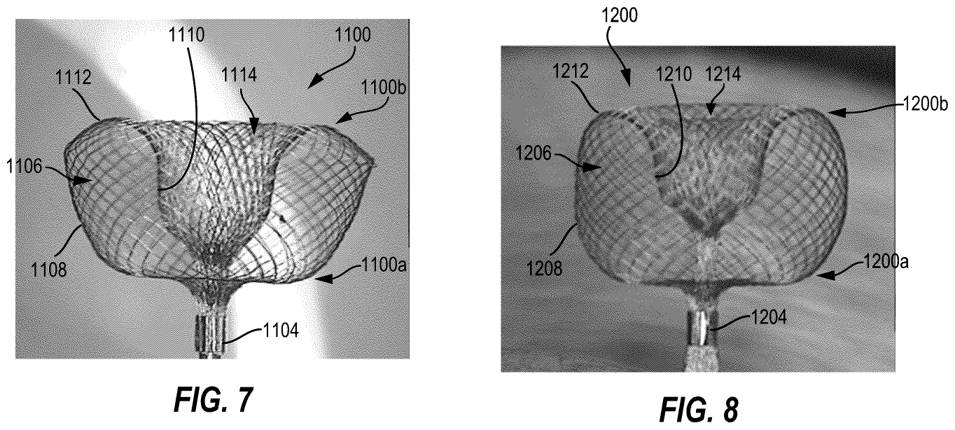

FIGS. 9A-9D show a series of fluoroscopic images at different stages of treating an aneurysm with the neck cover 400 in accordance with the present technology. FIG. 9A shows the neck cover 400400 positioned within an aneurysm A and in an expanded state. The neck cover 400 can be similar to any of the neck covers disclosed herein. As shown in FIG. 9A, the neck cover 400 can be positioned within the aneurysm A such that a proximal portion of the neck cover 400 is positioned at, adjacent to, and/or over the neck of the aneurysm A. At least a portion of the neck cover 400 can be positioned in contact with the wall of the aneurysm A. When the neck cover 400 is in the aneurysm A in an expanded state without an embolic composition, for example as shown in FIG. 9A, an outer layer 408 of the wall of the neck cover 400 can be spaced apart from the inner layer 410 of the wall of the neck cover 400. When an embolic composition 202 is being delivered to and/or delivered to the aneurysm A, for example as shown in FIGS. 9B and 9C, the embolic composition 202 can cause the inner layer 410 of the neck cover 400 to approach the outer layer 408. For example, as shown in FIG. 9D, when the aneurysm A is filled with embolic composition 202, the inner layer 410 of the wall can have a shape corresponding at least in part to a shape of the outer layer 408 of the wall. In some embodiments, the inner layer 410 can substantially conform to the first portion 1302. Additionally or alternatively, the inner layer 410 can remain spaced apart from the outer layer 408. As shown in FIG. 9C, when the aneurysm A has been occluded with the neck cover 400 and/or embolic composition 202, fluid flow into the aneurysm A is prevented and/or limited.

FIG. 10 is a side view of an example neck cover 400 in accordance with the present technology. The neck cover 400 may comprise a mesh having a proximal portion 400a configured to be positioned over a neck of the aneurysm and a distal portion 400b. In some embodiments, the neck cover 400 comprises a proximal coupler (not depicted in FIG. 4). The mesh may be biased towards a predetermined shape when the mesh is in an expanded, unconstrained state. The mesh can comprise a wall surrounding an interior region 406 and comprising a first portion 408, a second portion 410, and a third portion 412. The first portion 408 and the second portion 410 of the wall can each extend between the third portion 412 and the proximal coupler.

In an expanded state, an outer profile of the neck cover 400 defined by the first portion 408 can have any suitable shape for sufficiently spanning across and covering a neck of an aneurysm. In some embodiments, the outer profile of the neck cover 400 is substantially bowl shaped. For example, as shown in FIG. 10, the first portion 408 can comprise a first region 408a extending substantially laterally away from a proximal coupler and a second region 408b extending distally, and in some cases laterally, from the first region 408a to the third portion 412. The neck cover 400 and/or mesh can be substantially flat at the proximal portion 400a (e.g., the first region 408a of the first portion 408 of the wall) and/or may comprise a recessed portion at the proximal portion 400a. The recessed portion can surround all or a portion of the proximal coupler. In some embodiments, the neck cover 400 and/or mesh does not include a recessed portion at the proximal portion 400a. The second portion 410 of the neck cover 400 can define a cavity 414 when the neck cover 400 is in an expanded state. The cavity 414 can, for example, be bound by the second portion 410 of the wall. In some embodiments, the cavity 414 is substantially conical (e.g., see FIG. 10), cylindrical, or another suitable shape.

In an expanded state, the second portion 410 can be separated from the first portion 408 by a gap having a distance d. During use, an embolic composition can be delivered to an aneurysm sac via the cavity 414 of the neck cover 400. Delivery of the embolic composition can apply a radially outward pressure to the second portion 410 and/or a radially downward pressure to the third portion 412 to cause the second portion 410 to move closer to the first portion 408, thereby reducing the size of the gap between the first and second portions 408, 410 (e.g., reducing the distance d). Accordingly, movement of the second portion 410 towards the first portion 408 (and the reduction in the gap distance d) can correspond at least in part to an amount of an embolic composition delivered to the aneurysm and/or a degree of filling of the aneurysm. In embodiments in which the mesh comprises a plurality of radiopaque filaments, for example, movement of the second portion 410 can be visualized by a clinician to assess the occlusion of the aneurysm (e.g., degree of filling with an embolic composition, neck coverage, etc.). In some embodiments, an aneurysm is sufficiently filled with embolic composition when the second portion 410 of the wall of the neck cover 400 is positioned at and/or conforms to the first portion 408.

It may be advantageous for a neck cover of the present technology to be configured to deform upon introduction of an embolic composition to the neck cover and/or an aneurysm (or soon thereafter) such that visual feedback regarding a degree of aneurysm filling is provided throughout a greater duration of the process of delivering an embolic composition and/or at a high resolution. For example, when the neck cover 400 is in the expanded state, it may be advantageous for the distance d between the first and second portions 408, 410 to be large and/or for the cavity 414 to be small such that only a small amount of embolic composition needs to be delivered to the neck cover 400 and/or aneurysm before the second portion 410 begins deforming.

In various embodiments, the third portion 412 of the neck cover 400 may have a shape configured to facilitate deformation of the neck cover 400 in response to delivery of an embolic composition. For example, as shown in FIG. 10, the third portion 412 can extend along a wide, arcuate path between the first and second portions 408, 410 to facilitate radially outwardly directed movement of the second portion 410 towards the first portion 408 upon delivery of an embolic composition to the neck cover 400 and/or aneurysm.

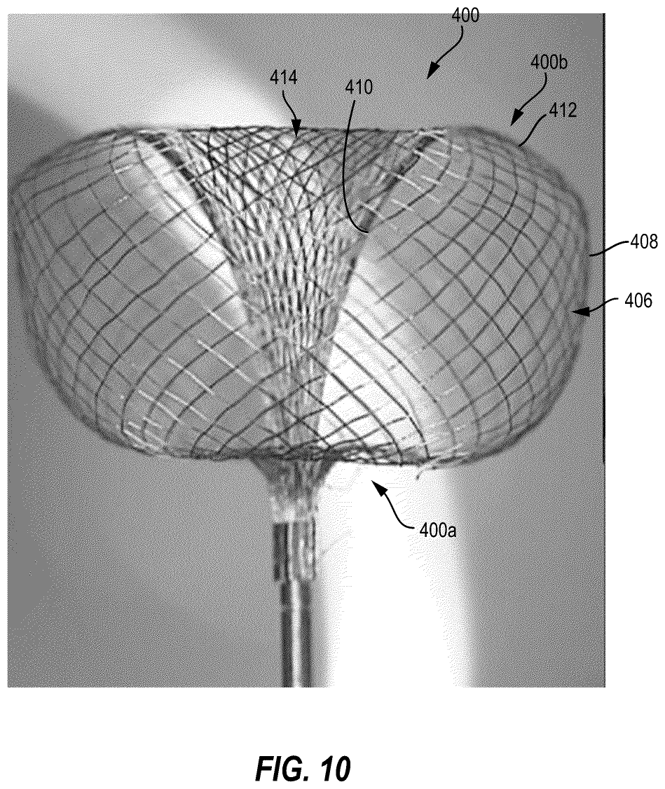

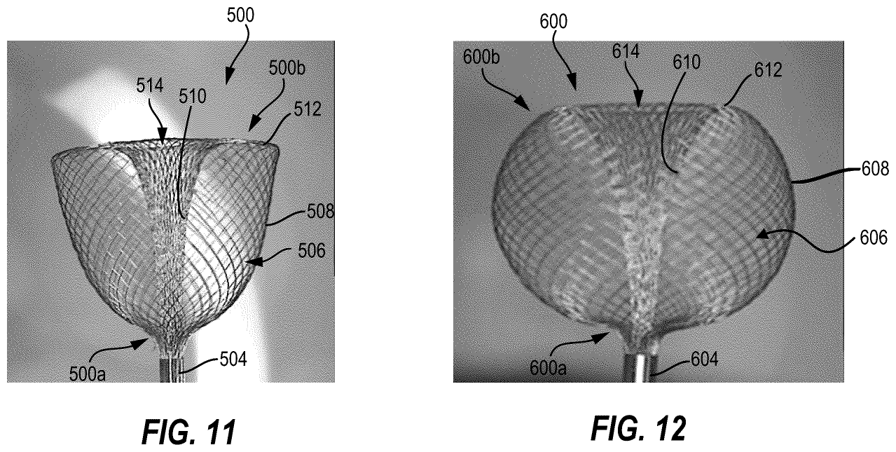

The neck covers 500, 600 of FIGS. 11 and 12 can be similar to the neck cover 400 shown in FIG. 4, except as detailed below. FIG. 11 is a side view of a neck cover 500 comprising a mesh having a proximal portion 500a, a distal portion 400b, and a wall surrounding an interior region 506 and comprising a first portion 508, a second portion 510, and third portion 512 extending therebetween. The first portion 508 and the second portion 510 of the wall can each extend between the third portion 512 and a proximal coupler 504. As shown in FIG. 11, in some embodiments the third portion 512 can extend between the first and second portions 508, 510 in a substantially lateral direction without extending proximally or distally to a substantial extent. In these and other embodiments, the third portion 512 can be substantially flat with a very large radius of curvature. The first portion 508 can extend between the third portion 512 and the proximal coupling such that the first portion 508 is concave towards a cavity 514 defined by the second portion 510. In these and other embodiments, the first portion 508 can extend distally and laterally from the proximal coupling to the third portion 512 such that an outer profile defined by the first portion 508 is substantially bowl-shaped. The outer profile can have any suitable shape for providing sufficient neck coverage, occlusion, and/or sealing of the aneurysm.

FIG. 12 is a side view of a neck cover 600 comprising a mesh having a proximal portion 600a, a distal portion 600b, and a wall surrounding an interior region 606 and comprising a first portion 608, a second portion 610, and a third portion 612 therebetween. In some embodiments, the third portion 612 can be substantially narrower and/or have a substantially smaller radius of curvature than the third portions 412, 512. The first portion 608 and the second portion 610 of the wall can extend between the third portion 612 and a proximal coupler 604. The first portion 608 can extend between the third portion 612 and the proximal coupler 604 such that the first portion 608 is concave towards a cavity 614 defined by the second portion 610. In some embodiments, an outer profile of the neck cover 600 defined by the first portion 608 is substantially spherical and/or globular. The first portion 608 can extend along an arcuate path such that a radially widest region of the first portion 608 is located at or near a center of the neck cover 600 and/or the first portion 608 tapers inwardly towards the proximal and distal ends 600a, 600b of the neck cover 600.

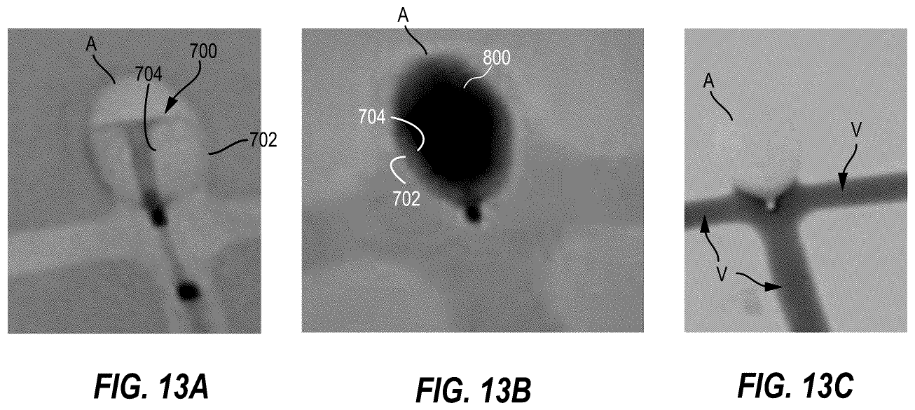

FIGS. 13A-13C show an example method of treating an aneurysm A with the neck cover 400. FIG. 13A shows the neck cover 400 positioned within the aneurysm A in an expanded state. The neck cover 400 can be similar to any of the neck covers disclosed herein. As shown in FIG. 13A, the neck cover 400 can be positioned within the aneurysm A such that the neck cover is positioned at, adjacent to, and/or over the neck of the aneurysm A. At least a portion of the neck cover 400 can be positioned in contact with the wall of the aneurysm A. When the neck cover 400 is in the aneurysm A in an expanded state without an embolic composition, for example as shown in FIG. 13A, a first portion 702 of the wall of the neck cover can be spaced apart from a second portion 704 of the wall of the neck cover 400. When the embolic composition 202 is positioned in the aneurysm, for example as shown in FIG. 13B, the embolic composition 202 can cause the second portion 704 of the wall of the neck cover 400 to approach the first portion 702. For example, as shown in FIG. 13B, when the aneurysm A is filled with embolic composition 202, the second portion 704 can conform to the first portion 702. FIG. 13C illustrates that, when the aneurysm A is occluded by the neck cover 400 and the embolic composition 202, fluid flow is blocked from adjacent vessels V into the aneurysm A.

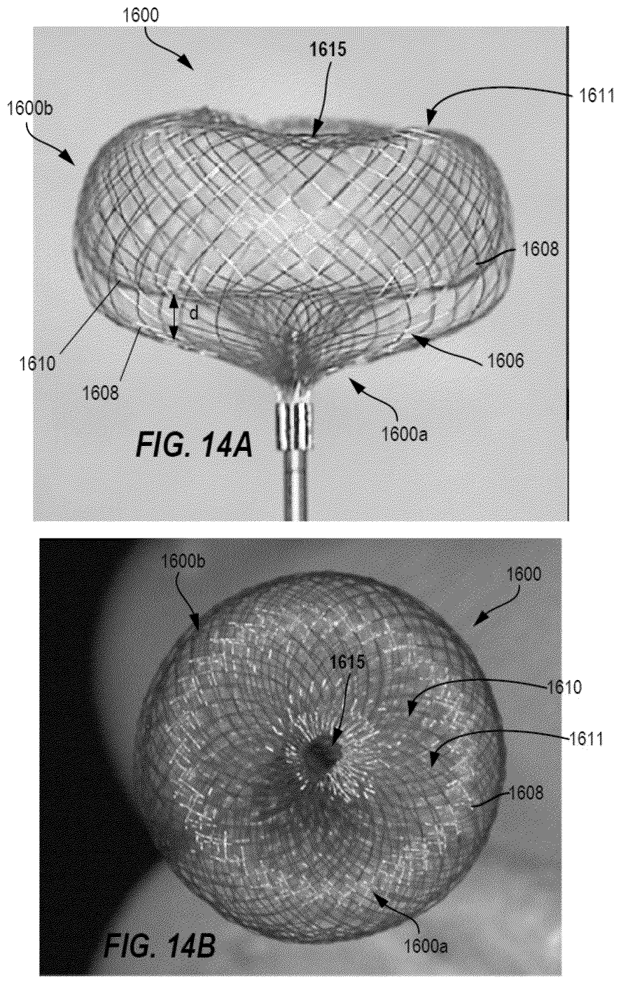

FIGS. 14A and 14B are perspective and side views, respectively, of a neck cover 1600 in accordance with the present technology. FIG. 14C is a cross-sectional view of the neck cover 1600 shown in FIGS. 14A and 14B. The neck cover 1600 can be similar to any of the neck covers disclosed herein, except as detailed below. The neck cover 1600 can comprise a mesh having a proximal portion 1600a configured to be positioned over a neck of the aneurysm and a distal portion 1600b. In some embodiments, the neck cover 1600 comprises a proximal coupler (not shown in FIGS. 14A and 14B). The mesh may be biased towards a predetermined shape when the mesh is in an expanded, unconstrained state. For example, as shown in FIGS. 14A and 14B, the mesh can be biased towards a disc and/or barrel shape (with a cavity extending therethrough) in the expanded state. The mesh can comprise a wall surrounding an interior region (not visible in FIGS. 14A and 14B) and comprising a first portion 1608, a second portion (not visible in FIGS. 14A and 14B), and a third portion 1612. The second portion can define a cavity 1614. In some embodiments, first portion 1608 and second portion have substantially similar contours. For example, as best shown in FIG. 14C, the first portion 1608 and second portion can have similar contours and are spaced apart by a distance d, which can be substantially constant along a length of the first portion 1608 and second portion. Additionally or alternatively, the first portion 1608 and second portion can have different contours.

In some embodiments, the first and second portions 1608, 16 first portion 1608 and second portion 10 each extend distally from the proximal coupler to the third portion 1612. In some embodiments, the first portion 1608 can comprise a radially outermost surface of the neck cover 1600 and the second portion can comprise a radially innermost surface of the neck cover 1600. Additionally or alternatively, as the first portion 1608 and/or second portion extend distally from the proximal coupler to the third portion 1612, the first portion 1608 and/or second portion can extend laterally away from a central longitudinal axis of the neck cover 1600 and then back towards the central longitudinal axis to the distal portion 1600b of the neck cover 1600. For example, as shown in FIGS. 14A and 14B, the neck cover 1600 can have a substantially flat distal surface 1611 formed by a distal region of the first portion 1608. The distal surface 1611 can have an opening 1615 extending therethrough that is defined by a fold 1630 where the inner 1610 and outer 1608 layers meet. In some embodiments, the opening 1615 can have a diameter that is ⅓ of the expanded diameter of the device. For example, a 6 mm diameter device would have a 2 mm diameter opening 1615.

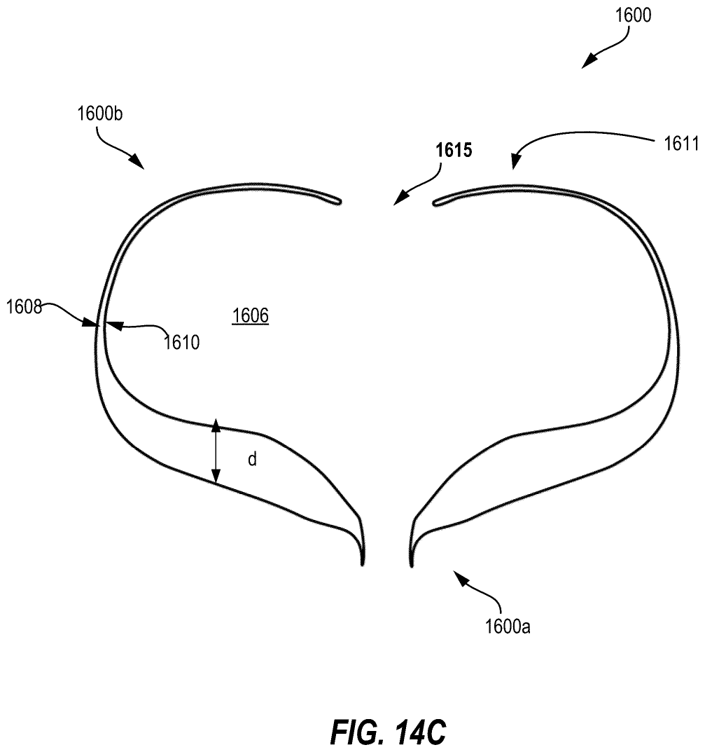

FIGS. 15A-15D depict a method for treating an aneurysm with the neck cover 1600. As shown, the embolic composition 202 is initially delivered to the cavity defined by the inner layer 1610. As the embolic composition 202 fills the space within the cavity, it begins to migrate through the opening 1615 at the distal surface 1611 and into the space in the aneurysm between the distal portion of the neck cover 1600 and the aneurysm dome. Because the inside of the cover 1600 fills with embolic composition 202 before the embolic composition reaches the dome and begins exerting a downward pressure, the distal surface 1611 of the cover 1600 remains substantially radially extending with embolic composition 202 both distal and proximal to it.

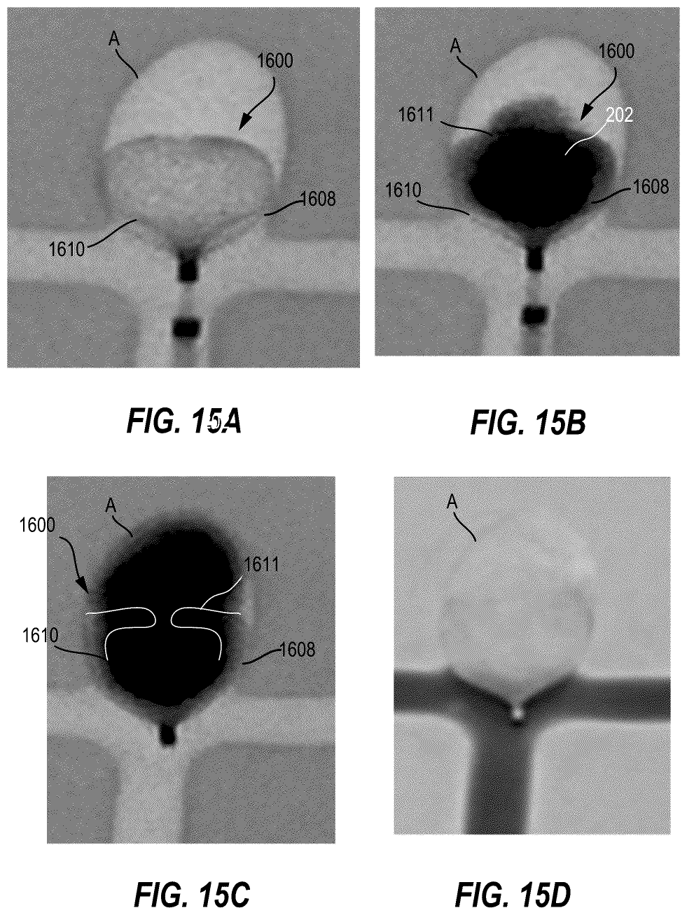

FIGS. 16A and 16B depict a neck cover 1800 in accordance with the present technology. The neck cover 1800 can be similar to any of the neck covers disclosed herein, except as detailed below. The neck cover 1800 can comprise a mesh having a proximal portion 1800a configured to be positioned over a neck of the aneurysm and a distal portion 1800b configured to be positioned within a cavity of an aneurysm. The mesh may be biased towards a predetermined shape when the mesh is in an expanded, unconstrained state. For example, as shown in FIGS. 16A and 16B, the mesh can be biased towards a bowl shape in the expanded state. The mesh can comprise a first portion 1808 and a second portion 1810 that meet at a fold 1812 defining the distal portion 1800b of the mesh. The first portion 1808 and the second portion 1810 can each comprise a free end and, in some embodiments, the free ends of the first and second portions 1808, 1810 can be secured to one another by a coupler 1814. In operation, the neck cover 1800 can be positioned across a neck of an aneurysm and assume an expanded state such that the mesh defines and surrounds a cavity 1816 that is concave towards the aneurysm dome. The coupler 1814 can be positioned within the cavity 1816 such that, when the neck cover 1800 is deployed in an aneurysm, the coupler 1814 does not protrude into the parent vessel.

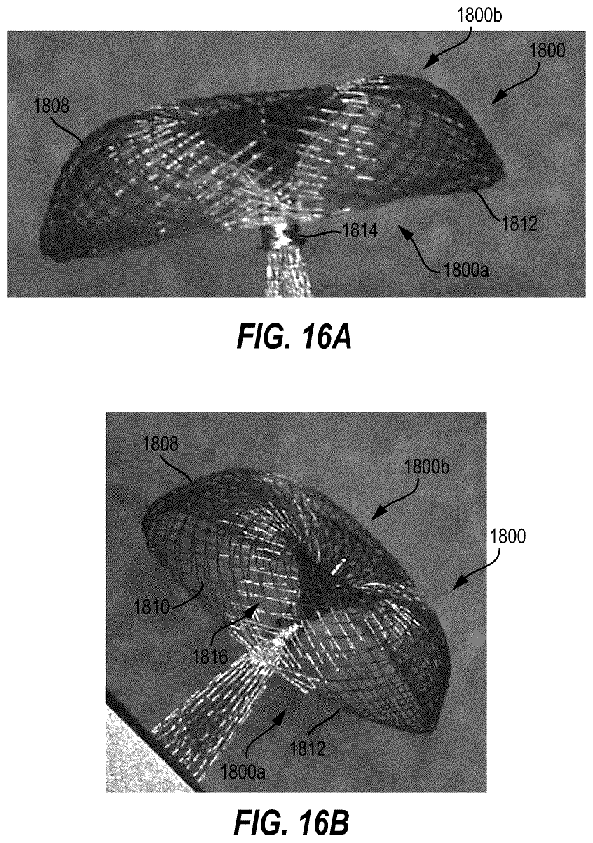

FIG. 17 shows a neck cover 1700 in accordance with the present technology comprising an aneurysm portion 1780 and an anchor portion 1782 that is configured to be positioned in the parent vessel such that the neck of the aneurysm is sandwiched between the bottom surface of the aneurysm portion 1780 and the top surface of the anchor portion 1782. In some embodiments the neck cover comprises a dual-layer braid and has an outer layer 1708 and an inner layer 1710. The anchor portion 1782 and the aneurysm portion 1780 can be continuous with one another and/or different parts of the same braid. For example, in some embodiments a single braid can be shaped to have a first flared portion (e.g., anchor portion 1782) and a second flared portion (e.g., aneurysm portion 1780) distal of the first flared portion along the longitudinal axis of the device.

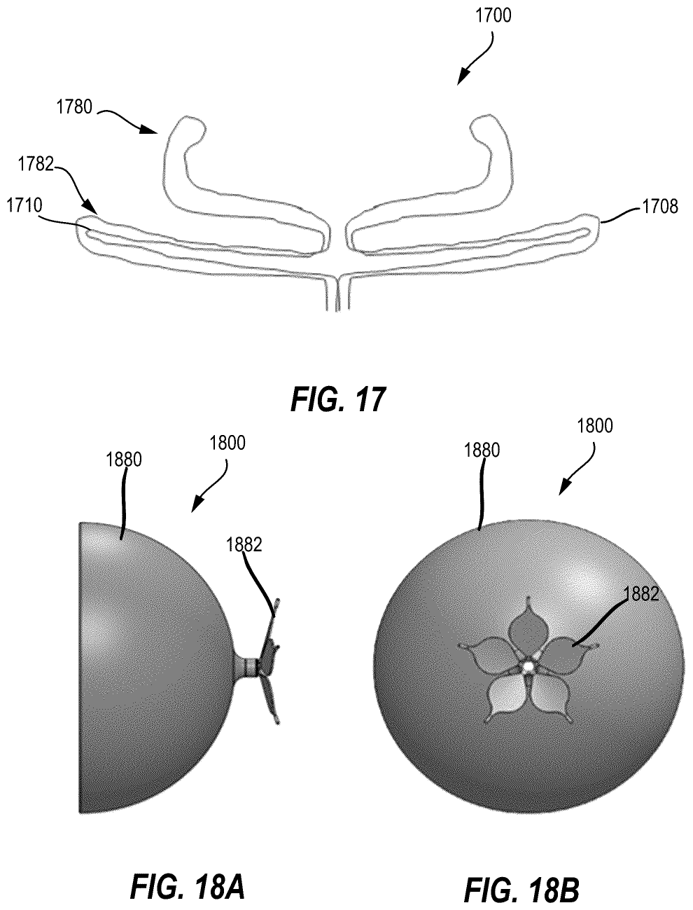

FIGS. 18A and 18B are different views of another neck cover that incorporates an anchor near the neck region. As shown in FIGS. 18A and 18B, the neck cover 1800 can include an aneurysm portion 1880 that is substantially cup-shaped (although it could have any shape) and an anchor portion 1882 that comprises a plurality of petals that extend radially away from a longitudinal axis of the device. In some embodiments, the aneurysm portion 1880 and the petals 1882 are formed of the same braid. The petals 1882 can be different groupings of the wire ends, for example.

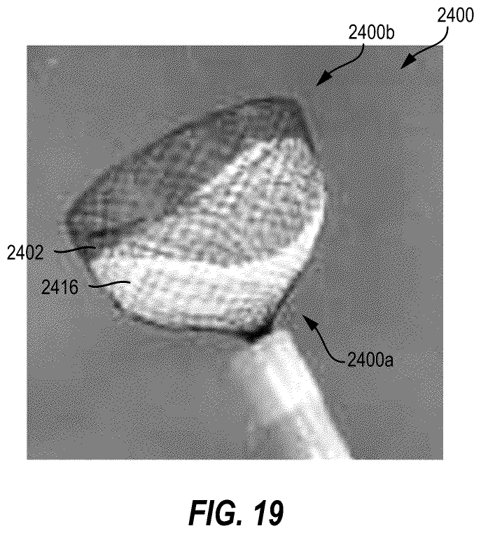

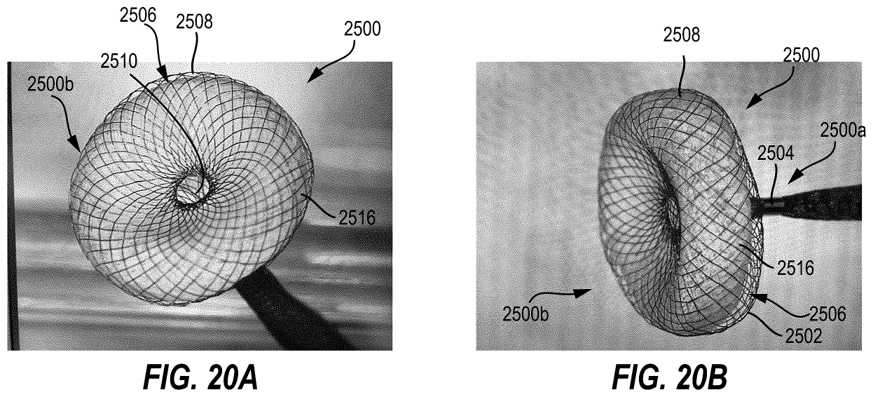

A neck cover in accordance with any of the embodiments disclosed herein can comprise a membrane configured to prevent or limit flow through the membrane and into an aneurysm. FIGS. 19-20B depict examples of such neck covers 2400, 2500. The neck cover 2400 shown in FIG. 19 can be similar to any of the neck covers disclosed herein, except as detailed below. The neck cover 2400 may comprise a mesh 2402 having a proximal portion 2400a configured to be positioned over a neck of the aneurysm and a distal portion 2400b configured to be positioned within a cavity of an aneurysm. In some embodiments, the neck cover 2400 comprises a proximal coupler (not depicted in FIG. 19). The mesh may be biased towards a predetermined shape when the mesh is in an expanded, unconstrained state. For example, as shown in FIG. 19, the neck cover 2400 can be substantially bowl shaped. The mesh can comprise a wall surrounding an interior region and comprising a first portion, a second portion, and a third portion such that the first portion and the second portion each extend between the third portion and the proximal coupler. The second portion can define a cavity of the neck cover 2400, which can be configured to receive an embolic composition therethrough.

As shown in FIG. 19, the neck cover 2400 can include a membrane 2416, which can be configured to enhance an occlusive property of the neck cover 2400. In some embodiments, the membrane 2416 can be positioned within the interior region between the first portion and the second portion of the mesh. By sandwiching the membrane 2416 between two portions of the wall of the mesh, the membrane 2416 can be secured to the mesh without additional steps such as welding, adhering, or otherwise coupling the membrane 2416 to the mesh. However, in some embodiments the membrane 2416 can be secured to the mesh via suturing, gluing, welding, crimping, or otherwise securing the membrane 2416 to the mesh. The membrane 2416 can be positioned between two or more portions of the mesh, on a radially inner surface of the mesh, on a radially outer surface of the mesh, on a proximal surface of the mesh, on a distal surface of the mesh, etc. The membrane 2416 can be positioned at the proximal portion 2400a of the mesh such that, when the neck cover 2400 is delivered to an aneurysm, the membrane 2416 spans across at least a portion of the neck of the aneurysm. The membrane 2416 can span an entire length of the neck cover 2400 from the proximal portion 2400a to the distal portion 2400b or the membrane 2416 may not extend to the distal portion 2400b. In some embodiments, the membrane 2416 is substantially fluid impermeable. Examples of suitable membrane 2416 materials metals, polymers, and others. For example, the membrane 2416 can comprise a fluoropolymer (e.g., PTFE, ePTFE, FEP, etc.) or another suitable polymer. In some embodiments, the membrane 2416 comprises a biological material (e.g., autologous tissue, cadaveric tissue, xenografts, etc.).

FIGS. 20A and 20B depict a neck cover 2500 comprising a mesh 2502 and a membrane 2516. The neck cover 2500 has a proximal portion 2500a configured to be positioned over a neck of the aneurysm and a distal portion 2500b configured to be positioned within a cavity of an aneurysm. The neck cover 2500 of FIGS. 20A and 20B has a substantially toroidal shape, which differs from the bowl shape of the neck cover 2400 of FIG. 19. Although FIGS. 19-20B depict neck covers having two specific shapes, a neck cover having any of the shapes disclosed herein can comprise a membrane as described with reference to FIGS. 19-20B.

The mesh 2502 shown in FIGS. 20A and 20B comprises a wall surrounding an interior region 2506 and comprising a first portion 2508, a second portion 2510, and a third portion 2512. In some embodiments, the neck cover 2500 includes a proximal coupler 2504. The first portion 2508 and the second portion 2510 of the wall can each extend between the third portion 2512 and the proximal coupler 2504. The second portion 2510 can define a cavity 2514 of the neck cover 2500, which can be configured to receive an embolic composition therethrough.

As shown in FIG. 20A, the membrane 2516 can be positioned in the interior region 2506 defined by the wall of the mesh 2502. In embodiments in which the second portion 2510 of the wall of the mesh 2502 is spaced apart from the first portion 2508, the membrane 2516 can optionally be positioned at and/or in apposition with the second portion 2510. The membrane 2516 may or may not be secured or adhered to the mesh 2502. For example, the membrane 2516 can be free floating within the interior region 2506. In another example, the membrane 2516 can be adhered to the second portion 2510 of the wall to prevent or limit migration or deformation of the membrane 2516 within the interior region 2506.

In some embodiments, a method of manufacturing the neck cover 2500 shown in FIGS. 20A and 20B can comprise conforming the mesh 2502 to a shape forming mold and subjecting the mesh 2502 and shape forming mold to a shape setting procedure (e.g., heat treatment, etc.) to cause the mesh 2502 to retain a desired shape (e.g., the toroidal shape shown in FIGS. 20A and 20B, any of the other shapes disclosed herein, etc.) in an expanded, unconstrained state. In some embodiments, the mesh 2502 is superelastic and/or resilient such that the mesh 2502 can be elastically deformed. Accordingly, an outer layer of the mesh 2502 (e.g., corresponding to the first portion 2508 of the wall of the mesh 2502) can be separated from an inner layer of the mesh 2502 (e.g., corresponding to the second portion 2510) to open up the interior volume 2506. The membrane 2516 can be positioned on and/or adhered to the second portion 2510 and/or the first portion 2508. The outer layer (e.g., the first portion 2508) can then be returned to its desired position relative to the inner layer (e.g., the second portion 2510) with the membrane 2516 positioned therebetween. The proximal coupler 2504 can then be secured to the mesh 2502, thereby encapsulating the membrane 2516 between portions of the mesh 2502.

### IV. CONCLUSION

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

To the extent any materials incorporated herein by reference conflict with the present disclosure, the present disclosure controls.

Unless otherwise indicated, all numbers expressing dimensions, percentages, or other numerical values used in the specification and claims, are to be understood as being modified in all instances by the term “about.” Accordingly, unless indicated to the contrary, the numerical parameters set forth in the specification and attached claims are approximations that may vary depending upon the desired properties sought to be obtained by the present technology. At the very least, and not as an attempt to limit the application of the doctrine of equivalents to the scope of the claims, each numerical parameter should at least be construed in light of the number of reported significant digits and by applying ordinary rounding techniques. Additionally, all ranges disclosed herein are to be understood to encompass any and all subranges subsumed therein. For example, a range of “1 to 10” includes any and all subranges between (and including) the minimum value of 1 and the maximum value of 10, i.e., any and all subranges having a minimum value of equal to or greater than 1 and a maximum value of equal to or less than 10, e.g., 5.5 to 10.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1-15. (canceled)

16. A device for treating an aneurysm comprising an expandable mesh configured to be positioned in a cerebral aneurysm, the mesh comprising at least two mesh layers and a membrane positioned between two of the at least two mesh layers.

17. The device of claim 16, wherein the mesh is a braid.

18. The device of claim 16, wherein the mesh has a bowl shape.

19. The device of claim 16, wherein the membrane is fluid impermeable.

20. The device of claim 16, wherein the membrane has a plurality of perforations.

21. The device of claim 16, wherein the membrane comprises a fluoropolymer.

22. The device of claim 16, wherein the at least two mesh layers comprise an inner layer and an outer layer that are continuous with one another at a distal end portion of the mesh.

23. An occlusive device for treating an aneurysm, the device comprising:
a mesh configured to be implanted in an aneurysm and comprising a proximal end portion, a distal end portion, and a longitudinal axis extending therebetween, the mesh defining an opening at the distal end portion and outer and inner layers that are continuous with one another distally at the distal end portion of the mesh and proximally at the proximal end portion of the mesh via a securing means,
wherein the mesh is configured to transition between a collapsed state for delivery to an aneurysm through an elongated shaft and an expanded, unconstrained state,
wherein, in the expanded, unconstrained state:
an inner surface of the outer layer and an inner surface of the inner layer are separated by a radial distance such that the inner surfaces of the outer and inner layers enclose a cavity, the inner surface of the inner layer having a first region and a second region extending from the first region, and wherein the first region extends proximally from the opening and is concave towards the inner surface of the outer layer, and wherein the second region is convex towards the inner surface of the outer layer.

24. The device of claim 23, wherein the mesh comprises a plurality of braided filaments.

25. The device of claim 24, further comprising a membrane positioned between the outer and inner layers.

26. The device of claim 25, wherein the membrane is fluid impermeable.

27. The device of claim 25, wherein the membrane has a plurality of perforations.

28. The device of claim 25, wherein the membrane comprises a fluoropolymer.

29. An occlusive device for treating an aneurysm, the device comprising:
a mesh configured to be implanted in an aneurysm and having a proximal end portion, a distal end portion, and a longitudinal axis extending therebetween, the mesh further comprising an opening at the distal end portion and outer and inner layers that meet distally at the distal end portion of the mesh and proximally at the proximal end portion of the mesh via a securing means,
wherein the mesh comprises a collapsed state for delivery through an elongated shaft to the aneurysm and an expanded, unconstrained state, and wherein, in the expanded, unconstrained state:
an inner surface of the outer layer and an inner surface of the inner layer are separated by a radial distance such that the inner surfaces of the outer and inner layers enclose a first cavity, and
the outer layer defines a diameter that increases then decreases along the longitudinal axis of the mesh between the proximal and distal end portions.

30. The device of claim 29, wherein the mesh comprises a plurality of braided filaments.

31. The device of claim 29, further comprising a membrane positioned between the outer and inner layers.

32. The device of claim 31, wherein the membrane is fluid impermeable.

33. The device of claim 31, wherein the membrane has a plurality of perforations.

34. The device of claim 31, wherein the membrane comprises a fluoropolymer.
